(19) United States
(12) Patent Application Publication
(10) Pub. No.: US 2011/0064678 A1
(43) Pub. Date: Mar. 17, 2011

(54) STYLING AGENTS GIVING A HIGH DEGREE OF HOLD

(75) Inventors: Thorsten Knappe, Schenefeld (DE); Sabine Albrechtsen, Elmshorn (DE); Uwe Bergemann, Hamburg (DE)

(73) Assignee: HENKEL AG & CO. KGAA, Dusseldorf, DE (DE)

(21) Appl. No.: 12/442,878

(22) PCT Filed: Aug. 16, 2007
(86) PCT No.: PCT/EP07/58492

§ 371 Date: Mar. 25, 2009

(30) Foreign Application Priority Data
Sep. 27, 2006 (DE) 102006045966.0

(51) Int. Cl. A61K 8/81; A61Q 5/06

(52) U.S. Cl. 424/47; 424/70.16

(57) ABSTRACT

An agent for the temporary deformation of keratinic fibers comprises, in a cosmetically acceptable carrier, a) at least one copolymer A formed from: at least one monomer A1 selected from acrylic acid, methacrylic acid, acrylic acid alkyl esters, and methacrylic acid alkyl esters; at least one monomer A2 selected from acrylic acid hydroxyalkyl esters and methacrylic acid hydroxyalkyl esters; and at least one monomer A3 selected from succinic acid monoalkyl esters and succinic acid dialkyl esters; and at least one film-forming and setting amphoteric polymer. A method for using the agent for the temporary deformation of hair, and an aerosol hair spray containing the agent and at least one propellant also are provided.

### CROSS-REFERENCE TO RELATED APPLICATION

This application is a U.S. National Stage entry under 35 U.S.C §371 based on International Application No. PCT/EP2007/058492, filed 16 Aug. 2007, which was published under PCT Article 21(2) and claims the benefit of the filing date of German Patent Application No 10 2006 0459660 filed 27 Sep. 2006, the disclosures of which applications are hereby incorporated herein by reference.

### FIELD OF THE INVENTION

The present invention relates in general to cosmetic compositions. In particular, the present invention relates to agents for the temporary deformation of keratinic fibers, containing a specific combination of polymers; to the use of said agents for the temporary deformation of keratinic fibers; and to aerosol hair sprays based on said agents.

### BACKGROUND OF THE INVENTION

“Keratin-containing” fibers are understood in principle as all animal hairs, e.g. wool, horsehair, angora wool, furs, feathers, and products or textiles produced therefrom. By preference, however, the keratinic fibers are human hairs.

An attractive-looking hairstyle is generally regarded these days as an indispensable element of a well-groomed appearance. Given the currents of fashion, more and more hairstyles regarded as chic are ones that, for many types of hair, can be constructed, or maintained for a longer period of time of up to several days, only with the use of setting ingredients. Hair treatment agents that serve for permanent or temporary shaping of the hair therefore play an important role. Temporary shaping actions that are intended to yield good hold without impairing the hair's healthy appearance, for example its shine, can be achieved, for example, using hair sprays, hair waxes, hair gels, hair foams, blow-dry waves, etc.

Corresponding agents for the temporary shaping usually contain synthetic polymers as a shaping component. Preparations that contain a dissolved or dispersed polymer can be applied onto the hair by means of propellant gases or by way of a pump mechanism. Hair gels and hair waxes in particular, however, are generally not applied directly onto the hair but rather distributed in the hair by means of a comb or one's hands.

The most important property of an agent for the temporary deformation of keratinic fibers, hereinafter also called a styling agent, is to impart the strongest possible hold to the treated fibers in the shape that is generated. If the keratinic fibers involved are human hairs, terms also used are a strong “hairstyle hold” or a high “degree of hold” of the styling agent. The hairstyle hold is determined substantially by the nature and quantity of the synthetic polymer used, although the further constituents of the styling agent can also have an influence. Also often desired, in addition to a high degree of hold, are flexibility, elasticity, and plasticity. Many commercially available styling agents, in particular aerosol hair sprays, are already notable for a high degree of hold. They often reach their limits, however, when faced with user demands for creating increasingly wild and extreme hairstyles and reliably fixing them in place.

Accordingly, it is desirable to provide an agent for the temporary deformation of keratinic fibers that is notable for a very high degree of hold, such that the flexibility, elasticity, and plasticity of the polymer film are retained. In addition, other desirable features and characteristics will become apparent from the subsequent summary and detailed description and the appended claims, taken in connection with the accompanying drawings and this background

### DESCRIPTION OF THE DRAWINGS

The present invention will hereinafter be described in conjunction with the following drawing figures, wherein like numerals denote like elements, and

FIG. 1 is an illustration of the structural formula of a linear dimethiconol that can be used in an embodiment of the present invention;

FIG. 2 is an illustration of the structural formula of a branched dimethiconol that can be used in an embodiment of the present invention;

FIG. 3 is an illustration of the structural formula of a linear dimethicone that can be used in an embodiment of the present invention;

FIG. 4 is an illustration of the structural formula of a branched dimethicone that can be used in an embodiment of the present invention;

FIG. 5 is an illustration of the structural formula of a dimethicone copolyol that can be used in an embodiment of the present invention;

FIG. 6 is an illustration of the structural formula of another dimethicone copolyol that can be used in an embodiment of the present invention;

FIG. 7 is an illustration of the structural formula of a branched dimethicone copolyol that can be used in an embodiment of the present invention;

FIG. 8 is an illustration of the structural formula of another branched dimethicone copolyol that can be used in an embodiment of the present invention;

FIG. 9 is an illustration of the structural formula of an aminofunctional silicone that can be used in an embodiment of the present invention;

FIG. 10 is an illustration of the structural formula of a particular aminofunctional silicone of FIG. 9 that can be used in an embodiment of the present invention;

FIG. 11 is an illustration of the structural formula of another particular aminofunctional silicone of FIG. 9 that can be used in an embodiment of the present invention;

FIG. 12 is an illustration of the structural formula of a further particular aminofunctional silicone of FIG. 9 that can be used in an embodiment of the present invention;

FIG. 13 is an illustration of the structural formula of cationic polymer that can be used in an embodiment of the present invention;

FIG. 14 is an illustration of the structural formula of monomers having quaternary ammonium groups that can be used to form amphoteric polymers useful in an embodiment of the present invention;

FIG. 15 is an illustration of the structural formula of monomeric carboxylic acids that can be used to form amphoteric polymers useful in an embodiment of the present invention;

FIG. 16 is an illustration of the structural formula of dicarboxylic acids useful in an embodiment of the present invention;

FIG. 17 is an illustration of the structural formula of an ectoin or ectoin derivative useful in an embodiment of the present invention;

FIG. 18 is an illustration of the structural formula of another ectoin or ectoin derivative useful in an embodiment of the present invention;

FIG. 19 is an illustration of the structural formula of fatty acid partial glycerides useful in an embodiment of the present invention; and

FIGS. 20-28 are illustrations of the structural formulas of cationic direct-absorbing dyes useful in embodiments of the present invention.

### BRIEF SUMMARY

An agent for the temporary deformation of keratinic fibers, a method for using the agent, and an aerosol hair spray comprising the agent are provided. In accordance with an exemplary embodiment of the present invention, the agent for the temporary deformation of keratinic fibers comprises, in a cosmetically acceptable carrier, at least one copolymer A formed from: at least one monomer A1 selected from the group consisting of acrylic acid, methacrylic acid, acrylic acid alkyl esters, and methacrylic acid alkyl esters; at least one monomer A2 selected from the group consisting of acrylic acid hydroxyalkyl esters and methacrylic acid hydroxyalkyl esters; and at least one monomer A3 selected from the group consisting of succinic acid monoalkyl esters and succinic acid dialkyl esters; and at least one film-forming and setting amphoteric polymer B.

A method for using an agent for the temporary deformation of hair is provided in accordance with an exemplary embodiment of the present invention. The method comprises the step of applying the agent to hair, wherein the agent comprises, in a cosmetically acceptable carrier, at least one copolymer A formed from: at least one monomer A1 selected from the group consisting of acrylic acid, methacrylic acid, acrylic acid alkyl esters, and methacrylic acid alkyl esters; at least one monomer A2 selected from the group consisting of acrylic acid hydroxyalkyl esters and methacrylic acid hydroxyalkyl esters; and at least one monomer A3 selected from the group consisting of succinic acid monoalkyl esters and succinic acid dialkyl esters; and at least one film-forming and setting amphoteric polymer B.

An aerosol hair spray also is provided in accordance with another exemplary embodiment of the present invention. The aerosol hair spray comprises an agent for the temporary deformation of keratinic fibers and at least one propellant. The agent comprises, in a cosmetically acceptable carrier, at least one copolymer A formed from: at least one monomer A1 selected from the group consisting of acrylic acid, methacrylic acid, acrylic acid alkyl esters, and methacrylic acid alkyl esters; at least one monomer A2 selected from the group consisting of acrylic acid hydroxyalkyl esters and methacrylic acid hydroxyalkyl esters; and at least one monomer A3 selected from the group consisting of succinic acid monoalkyl esters and succinic acid dialkyl esters; and at least one film-forming and setting amphoteric polymer B.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, an agent for the temporary deformation of keratinic fibers that is notable for a very high degree of hold, such that the flexibility, elasticity, and plasticity of the polymer film are retained, can be achieved by a combination of specific polymers.

In one exemplary embodiment of the present invention, an agent for the temporary deformation of keratinic fibers comprises, in a cosmetically acceptable carrier,
a) at least one copolymer A formed from:
   at least one monomer A1 comprising acrylic acid, methacrylic acid, acrylic acid alkyl esters, or methacrylic acid alkyl esters;
   at least one monomer A2 comprising acrylic acid hydroxyalkyl esters or methacrylic acid hydroxyalkyl esters; and
   at least one monomer A3 comprising succinic acid monoalkyl esters or succinic acid dialkyl esters; and
b) at least one film-forming and/or setting amphoteric polymer.

Copolymers A, and the use thereof as film-forming and/or setting polymers, are known. These copolymers are notable in particular for a very high degree of hold. It has been shown, however, that when they are used in usual formulations for styling agents, especially in combination with usual constituents of an aerosol hair spray, the high degree of hold of copolymers A is lost. It has now been found, surprisingly, that the high degree of hold of copolymers A is retained when they are utilized in combination with an amphoteric film-forming and/or setting polymer.

In an exemplary embodiment of the present invention, the agents for the temporary deformation of keratinic fibers contain at least one copolymer A.

In a preferred embodiment, the copolymer A is formed from:
at least one monomer A1 comprising acrylic acid, methacrylic acid, acrylic acid C₁ to C₁₀ alkyl esters, or methacrylic acid C₁ to C₁₀ alkyl esters;
at least one monomer A2 comprising acrylic acid hydroxy-C₁ to C₁₀ alkyl esters or methacrylic acid hydroxy-C₁ to C₁₀ alkyl esters; and
at least one monomer A3 comprising succinic acid mono-C₁ to C₆ alkyl esters or succinic acid di-C₁ to C₆ alkyl esters.

For purposes of the present invention, what are to be understood as “copolymers A formed from the aforesaid monomers” are only those copolymers that contain, in addition to polymer units that result from the incorporation of the aforesaid monomers A1, A2, and A3 into the copolymer, a maximum of 5 weight percent (wt %), preferably a maximum of 1 wt %, of polymer units that are attributable to the incorporation of other monomers. In a preferred embodiment, copolymers A are formed exclusively from polymer units that result from the incorporation of the aforesaid monomers A1, A2, and A3 into the copolymer.

In a preferred embodiment, monomers A1 comprise acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, and/or methacrylic acid isopropyl ester, or combinations thereof.

In another preferred embodiment, monomers A2 comprise hydroxymethyl acrylate, hydroxymethyl methacrylate, (2-hydroxyethyl)acrylate, (2-hydroxyethyl)methacrylate, (2-hydroxypropyl)acrylate, (2-hydroxypropyl) methacrylate, (3-hydroxypropyl)acrylate, (3-hydroxypropyl)methacrylate or combinations thereof.

In a further preferred embodiment, monomers A3 comprise methyl hydrogensuccinate, methyl succinate, ethyl hydrogensuccinate, ethyl succinate, propyl hydrogensuccinate, propyl succinate, isopropyl hydrogensuccinate, isopropyl succinate or combinations thereof. More preferably monomers A3 comprise methyl succinate and/or ethyl succinate.

In a more preferred embodiment, copolymer A is formed from:
at least one monomer A1 comprising acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, methacrylic acid isopropyl ester, or a combination thereof;
at least one monomer A2 comprising hydroxymethyl acrylate, hydroxymethyl methacrylate, (2-hydroxyethyl)acrylate, (2-hydroxyethyl)methacrylate, (2-hydroxypropyl)acrylate, (2-hydroxypropyl)methacrylate, (3-hydroxypropyl)acrylate, (3-hydroxypropyl)methacrylate, or a combination thereof; and
at least one monomer A3 comprising methyl succinate and/or ethyl succinate.

The copolymers A can be manufactured from the aforesaid monomers using known polymerization methods. Very particularly preferred copolymers A are the copolymers referred to under INCI nomenclature as Acrylates/C1-2 Succinates/Hydroxyacrylates Copolymer. These are commercially obtainable.

In one exemplary embodiment, the agent comprises the copolymer A in an about of from about 0.01 to about 20 wt % of the agent, preferably, the agent comprises the copolymer A in an amount of from about 0.05 to about 10 wt %, and more preferably comprises from about 0.1 to about 5 wt %, based on the entire hair setting agent.

In another exemplary embodiment, the agents for the temporary deformation of keratinic fibers contain at least one film-forming and/or setting amphoteric polymer B.

The film-forming and/or setting amphoteric polymer B comprises methacryloylbetaine/alkyl methacrylate copolymers, copolymers of monomers having carboxy and/or sulfone groups, in particular acrylic acid, methacrylic acid, itaconic acid, and/or monomers having amino groups, in particular monoalkylaminoalkyl acrylates, dialkylaminoalkyl acrylates, monoalkylaminoalkyl methacrylates, dialkylaminoalkyl methacrylates, monoalkylaminoalkyl acrylamides, dialkylaminoalkyl acrylamides, monoalkylaminoalkyl methacrylamides, dialkylaminoalkyl methacrylamides, and/or copolymers of N-octyl acrylamide, methyl methacrylate, hydroxypropyl methacrylate, N-tert.-butylaminoethyl methacrylate, and/or acrylic acid.

In a preferred embodiment of the present invention, the agent comprises as film-forming and/or setting amphoteric polymer B an N-octyl acrylamide/acrylic acid/tert.-butylaminoethyl methacrylate copolymer, such as the copolymer marketed by the National Starch Company of Bridgewater, N.J. under the designation Amphomer® (INCI name: Octylacrylamide/Acrylates/Butylaminoethyl Methacrylate Copolymer).

In an exemplary embodiment, the film-forming and/or setting amphoteric polymer B is present in the agent in an amount of from about 0.01 to about 20 wt % of the agent. Preferably the film forming and/or setting amphoteric polymer is present in the agent in an amount of from about 0.1 to about 15 wt % of the agent, and more preferably the film forming and/or setting amphoteric polymer is present in the agent in an amount of from about 1.0 to about 10 wt %, based on the entire hair setting agent. The agent may comprise, several film-forming and/or setting amphoteric polymers, although the total quantity of the film-forming and/or setting amphoteric polymers is preferably no more than about 20 wt %.

It has been shown that an optimum properties profile, in particular a particularly high degree of hold, is obtained when the agent comprises the copolymer A and the film-forming and/or setting amphoteric polymer B. In an exemplary embodiment, the agent comprises at a weight ratio from about 1:20 to about 1:1, preferably from about 1:20 to about 1:2, more preferably from about 1:20 to about 1:5.

In addition to copolymer A and the film-forming and/or setting amphoteric polymer B, the agents can comprise other known film-forming and/or setting polymers. These film-forming and/or setting polymers can be both permanently and temporarily cationic, anionic, or nonionic.

Because polymers are often multifunctional, their functions cannot always be clearly and unequivocally distinguished from one another. This applies in particular to film-forming and/or setting polymers. It is explicitly stated at this juncture, however, that in the context of the present invention, both film-forming and/or setting polymers are essential. Because the two properties are also not entirely independent of one another, the term “setting polymers” is also always understood to mean “film-forming polymers,” and vice versa.

Included among the preferred properties of the film-forming polymers is film formation. “Film-forming polymers” are to be understood as those polymers that, upon drying, leave behind a substantially continuous film on the skin, hair, or nails. Film-formers of this kind can be used in a very wide variety of cosmetic products such as, for example, face masks, make-up, hair setting agents, hair sprays, hair gels, hair waxes, hair therapies, shampoos, or nail polishes. Particularly preferred are those polymers that possess sufficient solubility in alcohol or in water/alcohol mixtures to be present in completely dissolved form in the agent. The film-forming polymers can be of synthetic or natural origin.

In one exemplary embodiment “film-forming polymers” are furthermore understood to be those polymers that, when used in about a 0.01 to about 20 wt % aqueous, alcoholic, or aqueous/alcoholic solution, are capable of depositing a transparent polymer film on the hair.

Suitable further synthetic film-forming, hair-setting polymers are, for example, homo- or copolymers that are constructed from at least one of the following monomers: vinylpyrrolidone, vinyl caprolactam, vinyl esters such as, for example, vinyl acetate, vinyl alcohol, acrylamide, methacrylamide, alkyl and dialkyl acrylamide, alkyl and dialkyl methacrylamide, alkyl acrylate, alkyl methacrylate, propylene glycol or ethylene glycol, the alkyl groups of these monomers preferably being C₁ to C₇ alkyl groups, more preferably C₁ to C₃ alkyl groups.

Mention may be made, by way of example, of homopolymers of vinylpyrrolidone or of N-vinylformamide. Further suitable synthetic film-forming, hair-setting polymers are, for example, copolymers of vinylpyrrolidone and vinyl acetate, terpolymers of vinylpyrrolidone, vinyl acetate, and vinyl propionate, polyacrylamides that are marketed, for example, under the commercial names Akypomine® P 191 by CHEM-Y of Germany, or Sepigel® 305 by Seppic of Fanfield, N.J.; polyvinyl alcohols that are marketed, for example, under the commercial names Elvanol® by DuPont of Wilmington, Del. or Vinol® 523/540 by the Air Products and Chemicals, Inc. of Allentown Pa., and polyethylene glycol/polypropylene glycol copolymers that are marketed, for example, under the commercial names Ucon® by Union Carbide Corporation of Houston, Tex.

Suitable natural film-forming polymers are, for example, cellulose derivatives, for example hydroxypropyl cellulose having a molecular weight from 30,000 to 50,000 g/mol, which is marketed for example under the commercial name Nisso SI® by Lehmann & Voss, Germany.

Setting polymers contribute to the hold, and/or to building up the hair volume and hair fullness, of the overall hairstyle. These so-called setting polymers are at the same time also film-forming polymers, and are therefore generally typical substances for shaping hair-treatment agents such as hair setting agents, hair foams, hair waxes, hair sprays. It is certainly possible for film formation to be localized, and for only a few fibers to be connected to one another.

Substances that furthermore impart hydrophobic properties to the hair are preferred in this context, since they decrease the hair's tendency to absorb moisture, i.e. water. This decreases loose hanging of strands of hair, and thus ensures long-term hairstyle construction and retention. The so-called “curl retention” test is often used as a test method for this. These polymeric substances can furthermore be successfully incorporated into leave-in and rinse-off hair therapies or shampoos. Because polymers are often multifunctional, i.e. exhibit multiple effects that are desirable in terms of applications engineering, numerous polymers fall into multiple groups categorized in terms of effect; this is also the case in the CFTA handbook.

In one exemplary embodiment, agents that comprise further film-forming and/or setting polymers comprise such polymers in an amount from about 0.01 to about 20 wt %, based on the entire hair setting agent. Preferably the polymers are present in an amount of from about 0.1 to about 15 wt % of the hair setting agent. Several film-forming and/or setting polymers can of course also be contained, although the total quantity of further film-forming and/or setting polymers is preferably at most 20 wt %. In a preferred embodiment, the agents comprise, as film-forming and/or setting polymers, exclusively copolymers A and film-forming and/or setting amphoteric polymers B.

The agents according to the present invention contain copolymers A and the film-forming and/or setting amphoteric polymers B in a cosmetically acceptable carrier. In one exemplary embodiment, the cosmetically acceptable carriers are aqueous, alcoholic, or aqueous/alcoholic media. In a preferred embodiment, the cosmetically acceptable carriers have at least about 10 wt % water based on the entire agent. In another embodiment, the cosmetically acceptable carriers comprise the lower alcohols having 1 to 4 carbon atoms usually used for cosmetic purposes, for example, ethanol and isopropanol.

In one exemplary embodiment, the cosmetically acceptable carrier comprises additional co-solvents, such as organic solvents, or a mixture of solvents having a boiling point under 400° C. In a preferred embodiment, the co-solvents are present in an amount of from about 0.1 to about 15 wt %, more preferably from about 1 to about 10 wt %, based on the entire agent. Unbranched or branched hydrocarbons such as pentane, hexane, isopentane, and cyclic hydrocarbons, such as cyclopentane and cyclohexane, are particularly suitable as additional co-solvents. Further particularly preferred water-soluble solvents are glycerol, ethylene glycol, and propylene glycol, in a quantity of up to 30 wt % based on the entire agent.

The agents preferably have a pH from 2 to 11. More preferably, the pH range is between 2 and 8. Unless otherwise noted, the indications regarding pH refer in this context, for purposes of this document, to the pH at 25° C.

The agent according to the present invention can furthermore contain the adjuvants and additives that are usually added to conventional styling agents. Care-providing substances may be mentioned in particular as suitable adjuvants and additives.

A silicone oil and/or a silicone gum can be added, for example, as a care-providing substance. In a particular embodiment of the invention, the agents contain at least one silicone oil and/or one silicone gum. Suitable silicone oils or silicone gums include, in particular, dialkyl- and alkylarylsiloxanes such as, for example, dimethylpolysiloxane and methylphenylpolysiloxane, as well as alkoxylated, quaternized, or even anionic derivatives thereof. Cyclic and linear polydialkylsiloxanes, alkoxylated and/or aminated derivatives thereof, dihydroxypolydimethylsiloxanes, and polyphenylalkylsiloxanes are preferred.

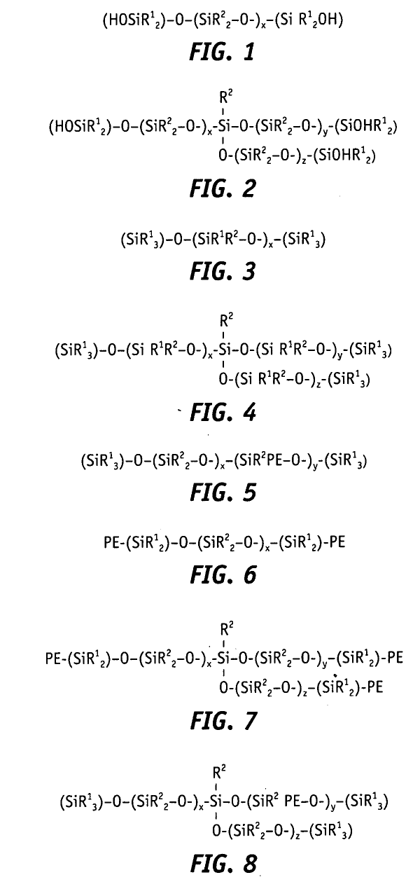

Silicone oils produce a wide variety of effects. For example, they simultaneously influence dry and wet combability, the feel of the dry and wet hair, and shine. The skilled artisan understands the term “silicone oils” to mean several structures of organosilicon compounds. It is understood firstly to mean the dimethiconols. These can be both linear and branched, and also cyclic or cyclic and branched. Linear dimethiconols can be represented by the structural formula of FIG. 1. Branched dimethiconols can be represented by the structural formula of FIG. 2.

The R¹ and R² radicals each denote, mutually independently, hydrogen, a methyl radical, a C₂ to C₃₀ linear, saturated or unsaturated hydrocarbon radical, a phenyl radical, and/or an aryl radical. Non-limiting examples of the radicals represented by R¹ and R² include alkyl radicals such as methyl, ethyl, propyl, isopropyl, butyl, isobutyl, pentyl, isopentyl, neopentyl, amyl, isoamyl, hexyl, isohexyl and the like; alkenyl radicals such as vinyl, halovinyl, alkylvinyl, allyl, haloallyl, alkylallyl; cycloalkyl radicals such as cyclobutyl, cyclopentyl, cyclohexyl, and the like; phenyl radicals, benzyl radicals, halogenated hydrocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl, and the like. Preferably, R¹ and R² are an alkyl radical that contains 1 to approximately 6 carbon atoms, and more preferably R¹ and R² are methyl. The numbers x, y, and z are whole numbers and range, mutually independently in each case, from 0 to 50,000. The molecular weights of the dimethiconols are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs, and more preferred viscosities are between 10,000 und 3,000,000 cPs. The most preferred range is between 50,000 und 2,000,000 cPs.

The following commercial products are recited as examples of suitable silicone oils: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all the aforesaid Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all the aforesaid Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all the aforesaid Crompton Corporation), SM555, SM2725, SM2765, SM2785 (all the aforesaid GE Silicones), Taylor T-Sil CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforesaid Wacker-Chemie GmbH).

Silicone oils suitable for use in the agents may also comprise dimethicones. Dimethicones can be both linear and branched, and also cyclic or cyclic and branched. Linear dimethicones can be represented by the following structural formula of FIG. 3. Branched dimethicones can be represented by the structural formula of FIG. 4.

The R¹ and R² radicals each denote, mutually independently, hydrogen, a methyl radical, a C₂ to C₃₀ linear, saturated or unsaturated hydrocarbon radical, a phenyl radical, and/or an aryl radical. Non-limiting examples of the radicals represented by R¹ and R² include alkyl radicals such as methyl, ethyl, propyl, isopropyl, butyl, isobutyl, pentyl, isopentyl, neopentyl, amyl, isoamyl, hexyl, isohexyl and the like; alkenyl radicals such as vinyl, halovinyl, alkylvinyl, allyl, haloallyl, alkylallyl; cycloalkyl radicals such as cyclobutyl, cyclopentyl, cyclohexyl, and the like; phenyl radicals, benzyl radicals, halogenated hydrocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl, and the like. In a preferred embodiment, R¹ and R² are an alkyl radical that contains 1 to approximately 6 carbon atoms, and more preferably R¹ and R² are methyl. The numbers x, y, and z are whole numbers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs, and more preferred viscosities are between 10,000 und 3,000,000 cPs. The most preferred range is between 50,000 und 2,000,000 cPs.

Dimethicone copolyols constitute a further group of silicones that are suitable. Dimethicone copolyols can be represented by the following structural formulas of FIG. 5 and FIG. 6. Branched dimethicone copolyols can be represented by the structural formula of FIG. 7 or by the structural formula of FIG. 8.

The R¹ and R² radicals each denote, mutually independently, hydrogen, a methyl radical, a C₂ to C₃₀ linear, saturated or unsaturated hydrocarbon radical, a phenyl radical, and/or an aryl radical. Non-limiting examples of the radicals represented by R¹ and R² include alkyl radicals such as methyl, ethyl, propyl, isopropyl, butyl, isobutyl, pentyl, isopentyl, neopentyl, amyl, isoamyl, hexyl, isohexyl and the like; alkenyl radicals such as vinyl, halovinyl, alkylvinyl, allyl, haloallyl, and alkylallyl; cycloalkyl radicals such as cyclobutyl, cyclopentyl, cyclohexyl, and the like; phenyl radicals, benzyl radicals, halogenated hydrocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl, and the like. In a preferred embodiment, R¹ and R² are an alkyl radical that contains 1 to approximately 6 carbon atoms, and more preferably R¹ and R² are methyl. PE denotes a polyoxyalkylene radical. Preferred polyoxyalkylene radicals are derived from ethylene oxide, propylene oxide, and glycerol. The numbers x, y, and z are whole numbers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs, and more preferred viscosities are between 10,000 und 3,000,000 cPs. The most preferred range is between 50,000 und 2,000,000 cPs. Corresponding dimethicone copolyols are commercially obtainable and are marketed, for example, by Dow Corning of Midland, Mich. under the designation Dow Corning® 5330 Fluid.

It will be understood that the dimethiconols, dimethicones, and/or dimethicone copolymers can already be present as an emulsion. The corresponding emulsion of the dimethiconols, dimethicones, and/or dimethicone copolyols can be manufactured both after manufacture of the corresponding dimethiconols, dimethicones, and/or dimethicone copolyols, from them and using usual emulsification methods known to the skilled artisan. For this purpose both cationic, anionic, nonionic, or zwitterionic surfactants and emulsifiers can be used, as auxiliaries, as adjuvants for manufacture of the corresponding emulsions. The emulsions of the dimethiconols, dimethicones, and/or dimethicone copolyols can of course also be manufactured directly by way of an emulsion polymerization method. Such methods, too, are very familiar to the skilled artisan.

In one exemplary embodiment, emulsions of the dimethiconols, dimethicones, and/or dimethicone copolyols comprise emulsified particles having a droplet size of from about 0.01 to about 10,000 μm, preferably from about 0.01 to about 100 μm, more preferably from about 0.01 to about 20 μm, and most preferably from about 0.01 to about 10 μm. The particle size is determined using the light-scattering method.

The terms “branched” dimethiconols, dimethicones, and/or dimethicone copolyols mean that the branching is greater than a random branching that occurs randomly as a result of contaminants in the respective monomers. “Branched” dimethiconols, dimethicones, and/or dimethicone copolyols are therefore to be understood, for purposes of the present invention, to mean that the degree of branching is greater than 0.01%. A degree of branching greater than 0.1% is preferred, and more preferably the degree of branching is greater than 0.5%. The degree of branching is determined from the ratio of unbranched monomers to the branching monomers, i.e. the quantity of tri- and tetrafunctional siloxanes. Both low-branching and high-branching dimethiconols, dimethicones, and/or dimethicone copolyols are suitable for use in the agents.

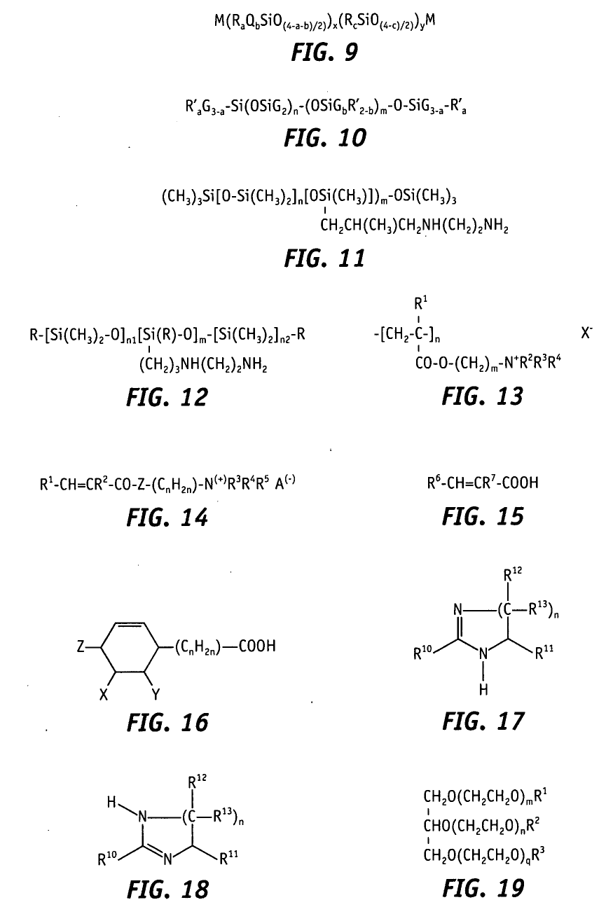

Suitable silicones are, in addition, aminofunctional silicones, in particular the silicones that are grouped under the INCI name Amodimethicone. These are to be understood as silicones that comprise at least one, optionally substituted, amino group. Such silicones can be described, for example, by the formula of FIG. 9, where: R is a hydrocarbon or hydrocarbon radical having 1 to approximately 6 carbon atoms; Q is a polar radical of the general formula —R¹Z, in which R¹ is a bivalent connecting group that is bound to hydrogen and to the Z radical, made up of carbon and hydrogen atoms, carbon, hydrogen, and oxygen atoms, or carbon, hydrogen, and nitrogen atoms, and Z is an organic aminofunctional radical that contains at least one aminofunctional group; “a” assumes values in the range from approximately 0 to approximately 2; “b” assumes values in the range from approximately 1 to approximately 3; “a”+“b” is less than or equal to about 3; and “c” is a number in the range of from approximately 1 to approximately 3; x is a number in the range from approximately 1 to approximately 2,000, preferably from approximately 3 to approximately 50, and more preferably from approximately 3 to approximately 25; y is a number in the range from approximately 20 to approximately 10,000, preferably from approximately 125 to approximately 10,000, and more preferably from approximately 150 to approximately 1,000; and M is a suitable silicone terminal group that is known in the existing art, for example, trimethylsiloxy. Non-limiting examples of the radicals represented by R include alkyl radicals such as methyl, ethyl, propyl, isopropyl, isopropyl, butyl, isobutyl, amyl, isoamyl, hexyl, isohexyl and the like; alkenyl radicals such as vinyl, halovinyl, alkylvinyl, allyl, haloallyl, and alkylallyl; cycloalkyl radicals such as cyclobutyl, cyclopentyl, cyclohexyl and the like; phenyl radicals, benzyl radicals, halocarbon radicals such as 3-chloropropyl, 4-bromobutyl, 3,3,3-trifluoropropyl, chlorocyclohexyl, bromophenyl, chlorophenyl, and the like, and sulfur-containing radicals such as mercaptoethyl, mercaptopropyl, mercaptohexyl, mercaptophenyl and the like; R is preferably an alkyl radical that contains 1 to approximately 6 carbon atoms, and R is more preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄—, and —(CH₂)₃C(O)SCH₂CH₂—.

Z is an organic aminofunctional radical containing at least one functional amino group. One possible formula for Z is NH(CH₂)_{z}NH₂, where z denotes a whole number from 1 to 50. Another possible formula for Z is —NH(CH₂)_{z}(CH₂)_{zz}NH, in which both z and zz, mutually independently, denote a whole number from 1 to 50; this structure encompasses diamino ring structures such as piperazinyl. Z is most preferably a —NHCH₂CH₂NH₂ radical. Another possible formula for Z is —N(CH₂)_{z}NX¹X² or NX¹X², in which X¹ and X² are selected, mutually independently in each case, from hydrogen and a hydrocarbon radical having approximately 1 to approximately 6 carbon atoms.

Very particularly preferably, Q denotes a polar aminofunctional radical of the formula —CH₂CH₂CH₂NHCH₂CH₂NH₂.

The molar ratio of the RₐQ_{b}SiO_{(4-a-b)/2} units to the R_{c}SiO_{(4-c)/2} units is in the range of from approximately 1:2 to 1:65, preferably from approximately 1:6 to approximately 1:65, and more preferably from approximately 1:15 to approximately 1:20. If one or more silicones of the above formulas are used, the different variable substituents in the above formula can be different in the different silicone components that are present in the silicone mixture.

Preferred aminofunctional silicones correspond to the formula of FIG. 10 where:
G is —H, a phenyl group, —OH, —O—CH₃, —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃;
a denotes a number between 0 and 3, in particular 0;
b denotes a number between 0 and 1, in particular 1,
m and n are numbers whose sum (m+n) is between 1 and 2000, preferably between 50 and 150, n preferably has a value of from about 0 to about 1999 and more preferably from about 49 to about 149, and m preferably has a value of from about 1 to about 2000, more preferably from about 1 to about 10;
R′ is a monovalent radical selected from:
   —N(R″)—CH₂—CH₂—N(R″)₂;
   —N(R″)₂;
   —N⁺(R″)₃A⁻;
   —N⁺H(R″)₂A⁻;
   —N⁺H₂(R″)A⁻; and
   —N(R″)—CH₂—CH₂—N⁺R″H₂A⁻;
   each R″ denoting identical or different radicals from the group of —H, phenyl, benzyl, the C₁₋₂₀ alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A representing an anion that is preferably selected from chloride, bromide, iodide, or methosulfate.

More preferred aminofunctional silicones correspond to the formula of FIG. 11 where m and n are numbers whose sum (m+n) is between about 1 and about 2000, preferably between about 50 and about 150; n preferably has a value from about 0 to about 1999 and more preferably from about 49 to about 149, and m preferably has a value of from about 1 to about 2000, more preferably from about 1 to about 10.

These silicones are referred to according to the INCI declaration as Trimethylsilylamodimethicone.

Also particularly preferred are aminofunctional silicones of the formula of FIG. 12 where R denotes —OH, —O—CH₃, or a —CH₃ group, and m, n1, and n2 are numbers whose sum (m+n1+n2) is between about 1 and about 2000, preferably between about 50 and about 150, the sum (n1+n2) preferably has a value of from about 0 to about 1999 and more preferably from about 49 to about 149, and m preferably has a value of from about 1 to about 2000, more preferably from about 1 to about 10.

These silicones are referred to according to the INCI declaration as Amodimethicone, and are obtainable, for example, in the form of an emulsion as the commercial product Dow Corning® 949, mixed with a cationic and a nonionic surfactant.

In a preferred embodiment, those aminofunctional silicones have an amine number above 0.25 meq/g, more preferably above 0.3 meq/g, and most preferably above 0.4 meq/g are. The amine number denotes the milliequivalent of amine per gram of the aminofunctional silicone; it can be ascertained by titration, and also indicated with the “mg KOH/g” unit.

Further suitable silicones include, for example:
oligomeric polydimethylcyclosiloxanes (INCI name: Cyclomethicone), in particular the tetrameric and the pentameric compound, which are marketed by Dow Corning as commercial products DC 245 Fluid, DC 344 or DC 345;
hexamethyldisiloxane (INCI name: Hexamethyldisiloxane), e.g. the product marketed under the designation Abil® K 520;
polyphenylmethylsiloxanes (INCI name: Phenyl Trimethicone), e.g. the commercial product DC 556 Cosmetic Grade Fluid of Dow Corning;
esters and partial esters of the silicone-glycol copolymers, such as those marketed, for example, by the Fanning Corporation, Chicago, Ill.; c under the commercial designation Fancorsil® LIM (INCI name: Dimethicone Copolyol Meadowfoamate); and
anionic silicone oils such as, for example, the product Dow Corning® 1784.

According to a preferred embodiment, the agent comprises at least two different silicone derivatives, more preferably a combination of a volatile and a non-volatile silicone. Those silicones that exhibit a volatility equal to or greater than the volatility of cyclic pentameric dimethylsiloxane are “volatile” for purposes of the invention. Such combinations are also available as commercial products (e.g. Dow Corning® 1401, Dow Corning® 1403, and Dow Corning® 1501, in each case mixtures of a cyclomethicone and a dimethiconol).

Preferred mixtures of different silicones are, for example, dimethicones and dimethiconols, linear dimethicones and cyclic dimethiconols. In a preferred embodiment, the mixture of silicones is made up of at least one cyclic dimethiconol and/or dimethicone, at least one further non-cyclic dimethicone and/or dimethiconol, and at least one aminofunctional silicone.

When different silicones are used as a mixture, the mixing ratio is largely variable. Preferably, however, all the silicones used for mixing are utilized at a ratio from 5:1 to 1:5 in the case of a binary mixture. A ratio from 3:1 to 1:3 is particularly preferred. Most preferred mixtures contain the silicones of the mixture very largely at a ratio of approximately 1:1, based in each case on the quantities utilized in wt %.

The agents contain the silicones preferably in quantities from 1 to 25 wt %, more preferably from 5 to 20 wt %, and most preferably from 7 to 15 wt %, based on the entire agent.

Although the agent preferably contains a silicone derivative as a care-providing substance, it is also possible for the agent to contain, instead of or in addition to a silicone component, at least one care-providing substance of another compound class. The agent can contain as a care-providing substance of another compound class, for example, at least one protein hydrolysate and/or one of its derivatives. Protein hydrolysates are product mixtures obtained by the acid-, base-, or enzyme-catalyzed breakdown of proteins. The term “protein hydrolysates” is also understood according to various embodiments of the present invention to mean total hydrolysates as well as individual amino acids and their derivatives, as well as mixtures of different amino acids. Polymers constructed from amino acids and amino-acid derivatives are also contemplated under the term “protein hydrolysates.” Included among the latter are, for example, polyalanine, polyasparagine, polyserine, etc. Further examples of suitable compounds are L-alanyl-L-proline, polyglycine, glycyl-L-glutamine, or D/L-methionine-S-methylsulfonium chloride. β-Amino acids and their derivatives, such as β-alanine, anthranilic acid, or hippuric acid, can of course also be used. The molecular weight of the protein hydrolysates is between about 75 (the molecular weight of glycine) and about 200,000 dalton; preferably the molecular weight is about 75 to about 50,000 dalton, and more preferably about 75 to about 20,000 dalton.

According to the present invention, protein hydrolysates of both plant and animal origin, or of marine or synthetic origin, can be used. Animal protein hydrolysates are, for example, elastin, collagen, keratin, silk, and milk protein hydrolysates, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm), and Kerasol® (Croda).

The use of silk protein hydrolysates is of particular interest. “Silk” is understood as the fibers of the cocoon of the mulberry silkworm (Bombyx mori L.). The raw silk fiber is made up of a double thread of fibroin. Sericin serves as a glue substance holding this double thread together. Silk is made up of 70 to 80 wt % fibroin, 19 to 28 wt % sericin, 0.5 to 1 wt % fat, and 0.5 to 1 wt % coloring agents and mineral constituents.

The essential constituents of sericin are approximately 46 wt % hydroxyamino acids. Sericin is made up of a group of 5 to 6 proteins. The essential amino acids of sericin are serine (Ser, 37 wt %), aspartate (Asp, 26 wt %), glycine (Gly, 17 wt %), alanine (Ala), leucine (Leu), and tyrosine (Tyr).

Water-insoluble fibroin is included among the scleroproteins having a long-chain molecular structure. The principal constituents of fibroin are glycine (44 wt %), alanine (26 wt %), and tyrosine (13 wt %). A further essential structural feature of fibroin is the hexapeptide sequence Ser-Gly-Ala-Gly-Ala-Gly.

It is technically simple to separate the two silk proteins from one another. It is therefore not surprising that both sericin and fibroin are known, each individually, as raw materials for use in cosmetic products. Protein hydrolysates and derivatives based on the respective individual silk proteins are also known raw materials in cosmetic agents. For example, sericin as such is marketed by Pentapharm Ltd. as a commercial product with the designation Sericin Code 303-02. Fibroin is offered far more frequently on the market as a protein hydrolysate, at various molecular weights. These hydrolysates are marketed in particular as “silk hydrolysates.” Hydrolyzed fibroin having average molecular weights between 350 and 1000 is marketed, for example, under the commercial designation Promois® Silk.

The positive properties of the silk protein derivatives from sericin and fibroin, individually for each one, are known in the literature. The cosmetic effects of sericin on the skin have been described as irritation-soothing, hydrating, and film-forming. The effect of a fibroin derivative is described, for example in DE 31 39 438 A1, as providing care to and revival of the hair. According to DE 102 40 757 A1, with the simultaneous use of sericin and fibroin, or derivatives and/hydrolysates thereof, it is furthermore possible to achieve a synergistic increase in the positive effects of the silk proteins and their derivatives.

In a preferred embodiment, therefore the agent comprises as a silk protein hydrolysate an active-substance complex (A) comprising the active substance (A1) selected from sericin, sericin hydrolysates, and/or derivatives thereof, as well as mixtures thereof, and an active substance (A2) selected from fibroin and/or fibroin hydrolysates and/or derivatives thereof and/or mixtures thereof. The active-substance complex (A) significantly improves, in synergistic fashion, the essential internal and external structural features presented above, and both the strength and elasticity of human hairs.

The following can be used as active substances (A1) in the active-substance complex (A):
native sericin;
hydrolyzed and/or further derivatized sericin, for example commercial products having the INCI names Sericin, Hydrolyzed Sericin, or Hydrolyzed Silk;
a mixture of the amino acids serine, aspartate, and glycine and/or the methyl, propyl, isopropyl, butyl, isobutyl esters thereof, the salts thereof such as, for example, hydrochlorides, sulfates, acetates, citrates, tartrates, such that the serine and/or derivatives thereof are contained in said mixture at about 20 to about 60 wt %, the aspartate and/or derivatives thereof at about 10 to about 40 wt %, and the glycine and/or derivatives thereof at about 5 to about 30 wt %, with the stipulation that the quantities of said amino acids and/or derivatives thereof by preference add up to 100 wt %; and
mixtures thereof.

The following can be used as active substances (A2) in the active-substance complex (A):
native fibroin converted into a soluble form;
hydrolyzed and/or further derivatized fibroin, especially partly hydrolyzed fibroin, which contains as a principal constituent the amino acid sequence Ser-Gly-Ala-Gly-Ala-Gly;
the amino acid sequence Ser-Gly-Ala-Gly-Ala-Gly;
a mixture of the amino acids glycine, alanine, and tyrosine and/or the methyl, propyl, isopropyl, butyl, isobutyl esters thereof, the salts thereof such as, for example, hydrochlorides, sulfates, acetates, citrates, tartrates, such that the glycine and/or derivatives thereof is contained in said mixture in quantities from about 20 to about 60 wt %, the alanine and derivatives thereof in quantities from about 10 to about 40 wt %, and the tyrosine and derivatives thereof in quantities from about 0 to about 25 wt %, with the stipulation that the quantities of said amino acids and/or derivatives thereof by preference add up to 100 w %; and
mixtures thereof.

Particularly good care-providing properties can be achieved if one of the two active-substance components of the active-substance complex (A) is used in the native or, if need be, solubilized form. It is also possible to utilize a mixture of several active substances (A1) and/or (A2).

It may be preferred to use the two active substances (A1) and (A2) in the agents at a ratio from about 10:90 to about 70:30, preferably about 15:85 to about 50:50, and more preferably about 20:80 to about 40:60, based on their respective active-substance contents.

The derivatives of the hydrolysates of sericin and fibroin encompass both anionic and cationized protein hydrolysates. The protein hydrolysates of sericin and fibroin, and the derivatives manufactured therefrom, can be obtained from the corresponding proteins by way of a chemical, in particular alkaline or acid, hydrolysis, by an enzymatic hydrolysis, and/or a combination of both types of hydrolysis. The hydrolysis of proteins results, as a rule, in a protein hydrolysate having a molecular weight distribution from approximately 100 dalton up to several thousand dalton. In an exemplary embodiment, those protein hydrolysates of sericin and/or fibroin and/or derivatives thereof have an underlying protein component with a molecular weight of from about 100 to about 25,000 dalton, preferably about 250 to about 10,000 dalton. Also to be understood as cationic protein hydrolysates of sericin and fibroin are quaternized amino acids and mixtures thereof. Quaternization of the protein hydrolysates or amino acids is often carried out by means of quaternary ammonium salts such as, for example, N,N-dimethyl-N-(n-alkyl)-N-(2-hydroxy-3-chloro-n-propyl)ammonium halides. The cationic protein hydrolysates can furthermore also be further derivatized. Typical examples that may be mentioned of cationic protein hydrolysates and derivatives usable in the agents are the following products listed under the INCI names in the “International Cosmetic Ingredient Dictionary and Handbook”, (seventh edition 1997, The Cosmetic, Toiletry, and Fragrance Association, 1101 17^{th} Street, N.W., Suite 300, Washington, D.C. 20036-4702), and available commercially: Cocodimonium Hydroxypropyl Hydrolyzed Silk, Cocodimonium Hydroxypropyl Silk Amino Acids, Hydroxypropyltrimonium Hydrolyzed Silk, Lauryldimonium Hydroxypropyl Hydrolyzed Silk, Steardimonium Hydroxypropyl Hydrolyzed Silk, Quaternium-79 Hydrolyzed Silk. Typical examples that may be mentioned of the anionic protein hydrolysates and derivatives according to the present invention are the following products listed under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702), and commercially available: Potassium Cocoyl Hydrolyzed Silk, Sodium Lauroyl Hydrolyzed Silk, or Sodium Stearoyl Hydrolyzed Silk. Lastly, the following products obtainable commercially under their INCI names may be mentioned as typical examples of the derivatives of sericin and fibroin usable according to the present invention: Ethyl Ester of Hydrolyzed Silk, and Hydrolyzed Silk PG-Propyl Methylsilanediol. Also usable are the commercially obtainable products having the INCI names Palmitoyl Oligopeptide, Palmitoyl Pentapeptide-3, Palmitoyl Pentapeptide-2, Acetyl Hexapeptide-1, Acetyl Hexapeptide-3, Copper Tripeptide-1, Hexapeptide-1, Hexapeptide-2, and MEA-Hydrolyzed Silk.

The effect of the active-substance complex (A) can be further enhanced by the addition of fatty substances. “Fatty substances” are to be understood as fatty acids, fatty alcohols, natural and synthetic waxes, which can be present both in solid form and in liquid form in aqueous dispersion, and natural and synthetic cosmetic oil components.

Protein hydrolysates of vegetable origin, e.g. soy, almond, bean, potato, and wheat protein hydrolysates, are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), and Crotein® (Croda).

Although the use of protein hydrolysates per se is preferred, it is also optionally possible to use instead of them, if applicable, amino-acid mixtures obtained in different fashion. It is likewise possible to use derivatives of protein hydrolysates, for example in the form of their fatty acid condensation products. Such products are marketed, for example, under the designations Lamepon® (Cognis), Lexein® (Inolex), Crolastin® (Croda), Crosilk® (Croda), or Crotein® (Croda). All isomeric forms, such as cis-trans isomers, diastereomers, and chiral isomers, are further contemplated herein. In an exemplary embodiment, a mixture of several protein hydrolysates are used.

The protein hydrolysates are contained in the agents, for example, in concentrations of from about 0.01 wt % to about 20 wt %, preferably from about 0.05 wt % to about 15 wt %, and more preferably in quantities from about 0.05 wt % to about 5 wt %, based in each case on the entire application preparation.

Cationic surfactants are further suitable as a care-providing substance of another compound class. In a preferred embodiment, cationic surfactants of the quaternary ammonium compound, esterquat, and amide amine types utilized. Preferred quaternary ammonium compounds are ammonium halides, in particular chlorides and bromides, such as alkyltrimethylammonium chlorides, dialkyldimethylammonium chlorides, and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, as well as the imidazolium compounds known under the INCI names Quaternium-27 and Quaternium-83. The long alkyl chains of the aforementioned surfactants preferably have 10 to 18 carbon atoms.

Esterquats are known substances that contain both at least one ester function and at least one quaternary ammonium group as structural elements. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. The products Armocare® VGH-70, an N,N-bis(2-palmitoyloxyethyl)dimethylammonium chloride, as well as Dehyquart® F-75, Dehyquart® C-4046, Dehyquart® L80, and Dehyquart® AU-35, are examples of such esterquats.

The alkylamidoamines are usually produced by amidation of natural or synthetic fatty acids and fatty acid cuts with dialkylaminoamines. A compound from this substance group that is particularly suitable according to the present invention is represented by the stearamidopropyldimethylamine available commercially under the designation Tegoamid® S 18.

The cationic surfactants are contained in the agents in quantities from about 0.05 to about 10 wt % based on the entire agent. Quantities from about 0.1 to about 5 wt % are particularly preferred.

Also suitable as a care-providing substance are care-providing polymers. Be it noted at this juncture that some care-giving polymers also exhibit film-forming and/or setting properties, and can therefore also be recited in the listing of suitable film-forming and/or setting polymers. A first group of care-providing polymers is the cationic polymers. “Cationic polymers” are to be understood herein as polymers that comprise in the main chain and/or side chain a group that can be “temporarily” or “permanently” cationic. According to various embodiments of the present invention, those polymers that possess a cationic group regardless of the pH of the agent are referred to as “permanently cationic.” These are, as a rule, polymers that contain a quaternary nitrogen atom, for example, in the form of an ammonium group. Preferred cationic groups are quaternary ammonium groups. In particular, those polymers in which the quaternary ammonium group is bound via a C₁₋₄ hydrocarbon group to a main polymer chain made up of acrylic acid, methacrylic acid, or their derivatives, have proven to be particularly suitable.

In a preferred embodiment, the cationic polymers are homopolymers of the general formula of FIG. 13 where R¹ is —H or —CH₃, R², R³ and R⁴ are selected, mutually independently, from C₁₋₄ alkyl, alkenyl, or hydroxyalkyl groups, m is the number 1, 2, 3 or 4, n is a natural number, and X⁻ is a physiologically acceptable organic or inorganic anion, as well as copolymers made up substantially of the monomer units presented in the formula of FIG. 13 as well as nonionogenic monomer units. In a preferred embodiment at least one of the following conditions applies,

R¹ denotes a methyl group,
R², R³ and R⁴ denote methyl groups, and
m has the value of 2.

Suitable as physiologically acceptable counterions X⁻ include, for example, halide ions, sulfate ions, phosphate ions, methosulfate ions, and organic ions such as lactate, citrate, tartrate, and acetate ions. Halide ions, in particular chloride, are preferred.

A particularly suitable homopolymer is the poly(methacryloyloxyethyltrimethylammonium chloride) (crosslinked, if desired) having the INCI name Polyquaternium-37. The crosslinking can be accomplished, if desired, with the aid of polyolefinically unsaturated compounds, for example divinylbenzene, tetraallyloxyethane, methylene bisacrylamide, diallyl ether, polyallylpolyglyceryl ether, or allyl ethers of sugars or sugar derivatives such as erythritol, pentaerythritol, arabitol, mannitol, sorbitol, sucrose, or glucose. Methylene bisacrylamide is a preferred cross-linking agent.

The homopolymer is preferably used in the form of a nonaqueous polymer dispersion that should comprise a polymer proportion not less than about 30 wt %. Such polymer dispersions are obtainable commercially under the designations Salcare® SC 95 (approx. 50% polymer proportion, further components: mineral oil (INCI name: Mineral Oil) and tridecylpolyoxypropylenepolyoxyethylene ether (INCI name: PPG-1-Trideceth-6)), and Salcare® SC 96 (approx. 50% polymer proportion, further components: mixture of diesters of propylene glycol with a mixture of caprylic and capric acid (INCI name: Propylene Glycol Dicaprylate/Dicaprate) and tridecylpolyoxypropylenepolyoxyethylene ether (INCI name: PPG-1-Trideceth-6)).

Copolymers having monomer units according to formula of FIG. 13 preferably contain acrylamide, methacrylamide, acrylic acid C₁₋₄ alkyl esters, and methacrylic acid C₁₋₄ alkyl esters as nonionogenic monomer units. Of these nonionogenic monomers, acrylamide is particularly preferred. These copolymers, as well as in the case of the homopolymers described above, can be crosslinked. A preferred copolymer is the crosslinked copolymer of acrylamide and methacryloyloxyethyltrimethyl-ammonium chloride. Such copolymers, in which the monomers are present at a weight ratio of approximately 20:80, are commercially obtainable, as an approximately 50% nonaqueous polymer dispersion, under the designation Salcare® SC 92.

Additional preferred cationic polymers include, for example:
quaternized cellulose derivatives such as those obtainable commercially under the designations Celquat® and Polymer JR®. The compounds Celquat® H 100, Celquat® L 200, and Polymer JR® 400 are preferred quaternized cellulose derivatives;
cationic alkylpolyglycosides according to DE Patent 44 13 686;
cationized honey, for example the commercial product Honeyquat® 50;
cationic guar derivatives such as, in particular, the products marketed under the trade names Cosmedia® Guar and Jaguar®;
polysiloxanes having quaternary groups, such as, for example, the commercially obtainable products Q2-7224 (manufacturer: Dow Corning; a stabilized trimethylsilylamodimethicone), Dow Corning® 929 Emulsion (containing a hydroxylamino-modified silicone that is also referred to as Amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80);
polymeric dimethyldiallylammonium salts and their copolymers with esters and amides of acrylic acid and methacrylic acid. The products available commercially under the designations Merquat® 100 (poly(dimethyldiallylammonium chloride)) and Merquat® 550 (dimethyldiallylammonium chloride/acrylamide copolymer) are examples of such cationic polymers;
copolymers of vinylpyrrolidone with quaternized derivatives of dialkylaminoalkyl acrylate and methacrylate, such as, for example, vinylpyrrolidone/dimethylaminoethyl methacrylate copolymers quaternized with diethyl sulfate. Such compounds are obtainable commercially under the designations Gafquat® 734 and Gafquat® 755;
vinylpyrrolidone/vinylimidazolium methochloride copolymers, such as those offered under the designations Luviquat® FC 370, FC 550, FC 905, and HM 552;
quaternized poly(vinylalcohol); and
the polymers known under the designations Polyquaternium-2, Polyquaternium-17, Polyquaternium-18, and Polyquaternium-27, having quaternary nitrogen atoms in the main polymer chain.

The polymers known under the designations Polyquaternium-24 (commercial product e.g. Quatrisoft® LM 200) can similarly be used as cationic polymers. Likewise usable according to the present invention are the copolymers of vinylpyrrolidone such as those available as the commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Additional suitable cationic polymers include are the so-called “temporarily cationic” polymers. These polymers usually contain an amino group that is present at certain pH values as a quaternary ammonium group and therefore cationically. Chitosan and its derivatives, such as those readily available commercially, for example, under the commercial designations Hydagen® CMF, Hydagen® HCMF, Kytamer® PC, and Chitolam® NB/101, are, for example, preferred.

In a preferred embodiment, the cationic polymers are cationic cellulose derivatives and chitosan and its derivatives, in particular the commercial products Polymer® JR 400, Hydagen® HCMF, and Kytamer® PC, cationic guar derivatives, cationic honey derivatives, in particular the commercial product Honeyquat® 50, cationic alkylpolyglycosides according to DE Patent 44 13 686, and polymers of the Polyquaternium-37 type.

Also to be listed among the suitable cationic polymers are cationized protein hydrolysates, in which context the underlying protein hydrolysate can derive from animals, for example from collagen, milk, or keratin, from plants, for example from wheat, corn, rice, potatoes, soy, or almonds, from marine life forms, for example from fish collagen or algae, or biotechnologically obtained protein hydrolysates. The protein hydrolysates serving as the basis for the cationic derivatives can be obtained from the corresponding proteins by way of a chemical, in particular alkaline or acid, hydrolysis, by an enzymatic hydrolysis, and/or a combination of both types of hydrolysis. The hydrolysis of proteins results, as a rule, in a protein hydrolysate having a molecular weight distribution from approximately 100 dalton up to several thousand dalton. In an exemplary embodiment, the agents comprise cationic protein hydrolysates having an underlying protein component with a molecular weight from about 100 to about 25,000 dalton, preferably about 250 to about 5,000 dalton. Also to be understood as cationic protein hydrolysates are quaternized amino acids and mixtures thereof. Quaternization of the protein hydrolysates or amino acids is often carried out by means of quaternary ammonium salts such as, for example, N,N-dimethyl-N-(n-alkyl)-N-(2-hydroxy-3-chloro-n-propyl)ammonium halides.

The cationic protein hydrolysates can also be further derivatized. Typical examples that may be mentioned of suitable cationic protein hydrolysates and derivatives are the following products listed under the INCI names in the “International Cosmetic Ingredient Dictionary and Handbook”, (seventh edition 1997, The Cosmetic, Toiletry, and Fragrance Association, 1101 17^{th} Street, N.W., Suite 300, Washington, D.C. 20036-4702), and available commercially: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, and Quaternium-79 Hydrolyzed Wheat Protein. The plant-based cationic protein hydrolysates and derivatives are very particularly preferred.

Amphoteric polymers used in preferred fashion are those polymerizates made up substantially of:

monomers having quaternary ammonium groups of the general formula of FIG. 14, where R¹ and R², mutually independently, denote hydrogen or a methyl group, and R³, R⁴ and R⁵, each mutually independently, denote an alkyl group having 1 to 4 carbon atoms, Z denotes an NH group or an oxygen atom, n is an integer from 2 to 5, and A⁽⁻⁾ is the anion of an organic or inorganic acid; and
monomeric carboxylic acids of the general formula of FIG. 15, where R⁶ and R⁷, mutually independently, denote hydrogen or a methyl group.

These compounds can be used according to the present invention both directly and in the form of salts that are obtained by neutralization of the polymerizates, for example using an alkaline hydroxide. Those polymerizates in which monomers of type (a) are used, in which R³, R⁴, and R⁵ are methyl groups, Z is an NH group, and A⁽⁻⁾ is a halide, methoxysulfate, or ethoxysulfate ion, are very particularly preferred; acrylamidopropyltrimethylammonium chloride is a particularly preferred monomer (a). Acrylic acid is preferably used as monomer (b) for the aforesaid polymerizates.

In an exemplary embodiment, the agents contain the care-providing cationic and/or amphoteric polymers in a quantity of from about 0.01 to about 5 wt %, and preferably in a quantity of from about 0.1 to about 2 wt %, based in each case on the entire agent.

The agent according to the present invention can furthermore contain at least one vitamin, provitamin, vitamin precursor, and/or derivative thereof as a care-providing substance. Those vitamins, provitamins, and vitamin precursors that are usually allocated to the groups A, B, C, E, F, and H are preferred.

The group of substances referred to as vitamin A includes retinol (vitamin A₁) as well as 3,4-didehydroretinol (vitamin A₂). β-Carotene is the provitamin of retinol. Suitable vitamin A components include, for example, vitamin A acid and its esters, vitamin A aldehyde, and vitamin A alcohol as well as its esters such as the palmitate and acetate. The agents contain the vitamin A component preferably in quantities of from about 0.05 to about 1 wt % based on the entire agent.

Members of the vitamin B group or vitamin B complex include, among others:
Vitamin B₁ (thiamine);
Vitamin B₂ (riboflavin);
Vitamin B₃. The compounds nicotinic acid and nicotinic acid amide (niacinamide) are often listed under this designation. Nicotinic acid amide is preferred. In an exemplary embodiment the agents comprise nicotinic acid amide in quantities of from about 0.05 to about 1 wt % based on the entire agent;
Vitamin B₅ (pantothenic acid, panthenol, and pantolactone). In the context of this group, panthenol and/or pantolactone are preferably used. Suitable derivatives of panthenol are, in particular, the esters and ethers of panthenol as well as cationically derivatized panthenols. Individual representatives are, for example, panthenol triacetate, panthenol monoethyl ether and its monoacetate, and cationic panthenol derivatives. In an exemplary embodiment, the aforesaid compounds of the vitamin B₅ type are contained in the agents in quantities of from about 0.05 to about 10 wt % based on the entire agent. Quantities of from about 0.1 to about 5 wt % are preferred; and
vitamin B₆ (pyridoxine as well as pyridoxamine and pyridoxal). In an exemplary embodiment, the aforesaid compounds of the vitamin B₆ type are contained in the agents in quantities of from about 0.01 to about 5 wt % based on the entire agent. Quantities of from about 0.05 to about 1 wt % are preferred.

In an exemplary embodiment, vitamin C (ascorbic acid) is utilized in the in quantities of from about 0.1 to about 3 wt % based on the entire agent. Utilization in the form of the palmitic acid ester, the glucosides or the phosphates are preferred. Utilization in combination with tocopherols likewise is preferred.

In an exemplary embodiment, vitamin E (tocopherols, in particular α-tocopherol) and its derivatives, which include in particular the esters such as the acetate, the nicotinate, the phosphate, and the succinate, are contained in the agents in quantities of from about 0.05 to about 1 wt % based on the entire agent.

In another exemplary embodiment, the agent comprise vitamin F. The term “vitamin F” is usually understood to mean essential fatty acids, in particular linoleic acid, linolenic acid, and arachidonic acid.

In an exemplary embodiment, the agents comprise vitamin H. This refers to (3aS,4S,6aR)-2-oxohexahydrothienol[3,4-d]-imidazole-4-valeric acid, for which the trivial name “biotin” has nevertheless since become established. In one embodiment biotin is contained in the agents in quantities of from about 0.0001 to about 1.0 wt %, preferably in quantities of from about 0.001 to about 0.01 wt %, based in each case on the entire agent.

The agents preferably contain vitamins, provitamins, and vitamin precursors from groups A, B, C, E and H. Panthenol, pantolactone, pyridoxine and its derivatives, as well as nicotinic acid amide and biotin, are particularly preferred.

D-panthenol, if applicable in combination with at least one of the aforementioned silicone derivatives, is also particularly preferably used as a care-providing substance.

In one exemplary embodiment the agents can contain at least one plant extract as a care-providing substance. These extracts are usually produced by extraction of the entire plant. In individual cases, however, it may also be preferred to produce the extracts exclusively from blossoms and/or leaves of the plant. With regard to plant extracts suitable for use in the agents, reference is made in particular to the extracts that are listed in the table beginning on page 44 of the 3rd edition of the Guideline for Declaring the Contents of Cosmetic Agents [Leitfaden zur Inhaltsstoffdeklaration kosmetischer Mittel], published by the Association of the personal hygiene and washing agents industry [Industrieverband Körperpflegeund Waschmittel e.V. (IKW)], Frankfurt. The extracts from green tea, oak bark, nettle, hamamelis, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root are preferred. Of these, the extracts from green tea, oak bark, nettle, hamamelis, hops, chamomile, burdock root, horsetail, linden blossoms, almond, aloe vera, coconut, mango, apricot, lemon, wheat, kiwi fruit, melon, orange, grapefruit, salvia, rosemary, birch, lady's-smock, wild thyme, yarrow, restharrow, meristem, ginseng, and ginger root are more preferred and extracts from green tea, almond, aloe vera, coconut, mango, apricot, lemon, wheat, kiwi fruit, and melon are most preferred.

Water, alcohols, and mixtures thereof can be used as extraction agents for manufacturing the aforesaid plant extracts. Among the alcohols, lower alcohols such as ethanol and isopropanol, but in particular polyvalent alcohols such as ethylene glycol and propylene glycol, both as the only extraction agent and mixed with water, are preferred. Plant extracts based on water/propylene glycol at a ratio from 1:10 to 10:1 have proven particularly suitable. The plant extracts can be used in both pure and diluted form. If they are used in diluted form, they usually contain approx. 2 to 80 wt % active substance, and contain as a solvent the extraction agent or extraction agent mixture used to obtain them. It may furthermore be preferred to use in the agents mixtures of several, in particular two, different plant extracts.

A number of carboxylic acids are also suitable as a care-providing substance. Short-chain carboxylic acids can be particularly advantageous for use in the agents. Short-chain carboxylic acids and their derivatives are understood herein to be carboxylic acids that can be saturated or unsaturated and/or straight-chain or branched or cyclic and/or aromatic and/or heterocyclic, and have a molecular weight below 750. Saturated or unsaturated straight-chain or branched carboxylic acids having a chain length of 1 to 16 carbon atoms in the chain are preferred; those having a chain length of 1 to 12 carbon atoms in the chain are more preferred.

The short-chain carboxylic acids can comprise one, two, three, or more carboxy groups. Carboxylic acids having multiple carboxy groups, in particular di- and tricarboxylic acids, are preferred. The carboxy groups can be present entirely or partly as an ester, acid anhydride, lactone, amide, imidic acid, lactam, lactim, dicarboximide, carbohydrazide, hydrazone, hydroxam, hydroxime, amidine, amide oxime, nitrile, or phosphonic or phosphate ester. The carboxylic acids can, of course, be substituted along the carbon chain or the ring structure. Substituents of the carboxylic acids include, for example, C₁ to C₈ alkyl, C₂ to C₈ alkenyl, aryl, aralkyl and aralkenyl, hydroxymethyl, C₂ to C₈ hydroxyalkyl, C₂ to C₈ hydroxyalkenyl, aminomethyl, C₂ to C₈ aminoalkyl, cyano, formyl, oxo, thioxo, hydroxy, mercapto, amino, carboxy or imino groups. Preferred substituents are C₁ to C₈ alkyl, hydroxymethyl, hydroxy, amino and carboxy groups. Substituents in the α-position are particularly preferred. Very particularly preferred substituents are hydroxy, alkoxy, and amino groups, in which context the amino function can be further substituted, if application, with alkyl, aryl, aralkyl, and/or alkenyl radicals. Furthermore, the phosphonic and phosphate esters are likewise preferred carboxylic acid derivatives.

The following may be mentioned as examples of carboxylic acids suitable for the use in the agents: formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-napthalenepentaacetic acid, malonaldehydic acid, 4-hydroxyphthalamidic acid, 1-pyrazolecarboxylic acid, gallic acid, or propanetricarboxylic acid, a dicarboxylic acid selected from the group formed by compounds of the general formula of FIG. 16, where Z denotes a linear or branched alkyl or alkenyl group having 4 to 12 carbon atoms, n is a number from 4 to 12, and one of the two groups X and Y denotes a —COOH group and the other hydrogen a methyl or ethyl radical, dicarboxylic acids of the general formula of FIG. 16 that additionally bear 1 to 3 methyl or ethyl substituents on the cyclohexene ring, as well as dicarboxylic acids resulting from the dicarboxylic acids according to formula of FIG. 16, in formal terms, by the attachment of one molecule of water to the double bond in the cyclohexene ring.

Dicarboxylic acids of formula of FIG. 16 are known in the literature. A manufacturing method may be inferred, for example, from U.S. Pat. No. 3,753,968. The dicarboxylic acids of the formula of FIG. 16 can be produced, for example, by reacting polyunsaturated dicarboxylic acids with unsaturated monocarboxylic acids in the form of a Diels-Alder cyclization. It is usual to proceed from a polyunsaturated fatty acid as a dicarboxylic acid component. Linoleic acid, accessible from natural fats and oils, is preferred. Acrylic acid in particular, but also e.g. methacrylic acid and crotonic acid, is preferred as a monocarboxylic acid component. Diels-Alder reactions usually result in isomer mixtures in which one component is present in excess. Both these isomer mixtures and the pure compounds can be used.

Also usable, in addition to the preferred dicarboxylic acids according to formula of FIG. 16, are those dicarboxylic acids that differ from the compounds according to formula of FIG. 16 by having 1 to 3 methyl or ethyl substituents on the cyclohexyl ring, or are made up from those compounds in formal terms by the attachment of one molecule of water to the double bond of the cyclohexene ring.

The dicarboxylic acid (mixture) resulting from the reaction of linoleic acid with acrylic acid has proven to be particularly effective for use in the agents. This is a mixture of 5- and 6-carboxy-4-hexyl-2-cyclohexene-1-octanoic acids. Such compounds are commercially available under the designations Westvaco Diacid® 1550 and Westvaco Diacid® 1595 (manufacturer: Westvaco).

In addition to the short-chain carboxylic acids listed above, their physiologically acceptable salts can also be used. Examples of such salts are the alkali, alkaline-earth, and zinc salts, as well as ammonium salts, among which the mono-, di-, and trimethyl-, ethyl-, and hydroxyethylammonium salts are also contemplated herein. Preferably, however, acids neutralized with alkaline-reacting amino acids, for example arginine, lysine, ornithine, and histidine, are used in the agents. It can also be preferred for formulation reasons to select the carboxylic acid from the water-soluble representative, in particular the water-soluble salts.

In a preferred embodiment, the carboxylic acid is 2-pyrrolidinone-5-carboxylic acid and its derivatives as a carboxylic acid. Particularly preferred are the sodium, potassium, calcium, magnesium or ammonium salts, in which context the ammonium ion carries, in addition to hydrogen, one to three C₁ to C₄ alkyl groups. The sodium salt is very particularly preferred. In an embodiment, the quantities used in the agents are in the range of from about 0.05 to about 10 wt % based on the entire agent, preferably from about 0.1 to about 5 wt %, and more preferably from about 0.1 to about 3 wt %.

In another embodiment, the carboxylic acid is a hydroxycarboxylic acid, especially the dihydroxy-, trihydroxy- and polyhydroxycarboxylic acids, as well as the dihydroxy-, trihydroxy- and polyhydroxydi, tri- and polycarboxylic acid. It has been found in this context that in addition to the hydroxycarboxylic acids, the hydroxycarboxylic acid esters, as well as mixtures of hydroxycarboxylic acids and their esters, and also polymeric hydroxycarboxylic acids and their esters, also can be utilized. Preferred hydroxycarboxylic acid esters are, for example, full esters of glycolic acid, lactic acid, malic acid, tartaric acid, or citric acid. Additional hydroxycarboxylic acid esters that are suitable in principle are esters of 8-hydroxypropionic acid, of tartronic acid, of D-gluconic acid, of saccharic acid, of mucic acid, or of glucuronic acid. Suitable as alcohol components of these esters are primary, linear or branched aliphatic alcohols having 8 to 22 carbon atoms, i.e. for example fatty alcohols or synthetic fatty alcohols. The esters of C₁₂ to C₁₅ fatty alcohols are particularly preferred in this context. Esters of this type are obtainable commercially, e.g. under the trademark Cosmacol® of EniChem, Augusta Industriale. Particularly preferred polyhydroxypolycarboxylic acids are polylactic acid and polytartaric acid as well as their esters.

Ectoin or ectoin derivatives, allantoin, taurine, and/or bisabolol are, furthermore, suitable as a care-providing substance. The term “ectoin and ectoin derivatives” is understood, according to various embodiments of the present invention, to mean compounds of the formula of FIG. 17 or of the formula of FIG. 18 and/or their physiologically acceptable salts and/or an isomeric or stereoisomeric form, in which:

R¹⁰ denotes a hydrogen atom, a branched or unbranched C₁ to C₄ alkyl radical, or a C₂ to C₄ hydroxyalkyl radical;
R¹¹ denotes a hydrogen atom, a —COOR¹⁴ grouping, or a —CO(NH)R¹⁴ grouping, in which context R¹⁴ can denote a C₁ to C₄ alkyl radical, an amino acid radical, or a dipeptide or tripeptide radical;
R¹² and R¹³ denote, mutually independently, a hydrogen atom, a C₁ to C₄ alkyl radical, or a hydroxy group, with the stipulation that the two radicals must not simultaneously denote a hydroxy group; and
n denotes an integer from 1 to 3.

Suitable physiologically acceptable salts of the general compounds according to the formulas of FIG. 17 or FIG. 18 are, for example, the alkaline, alkaline-earth, ammonium, triethylamine, or tris-(2-hydroxyethyl)amine salts, as well as those that result from the reaction of compounds according to the formulas of FIG. 17 or FIG. 18 with inorganic and organic acids such as hydrochloric acid, phosphoric acid, sulfuric acid, branched or unbranched, substituted or unsubstituted (for example with one or more hydroxy groups) C₁ to C₄ mono- or dicarboxylic acids, aromatic carboxylic acids and sulfonic acids such as acetic acid, citric acid, benzoic acid, maleic acid, fumaric acid, tartaric acid, and p-toluenesulfonic acid. Examples of particularly preferred physiologically acceptable salts are the Na, K, Mg, Ca, and ammonium salts of the compounds according to the formulas of FIG. 17 or FIG. 18, as well as the salts that result from the reaction of compounds according to the formulas of FIG. 17 or FIG. 18 with hydrochloric acid, acetic acid, citric acid, and benzoic acid.

Isomeric or stereoisomeric forms of the compounds according to the formulas of FIG. 17 and FIG. 18 are understood herein to mean all optical isomers, diastereomers, racemates, zwitterions, cations, or mixtures thereof that occur.

The term “amino acid” is understood to mean the stereoisomeric forms, e.g. D- and L-forms, of the following compounds:

asparagine, arginine, asparagic acid, glutamine, glutamic acid, β-alanine, γ-aminobutyrate, N_{ε}-acetyllysine, N_{δ}-acetylornithine, N_{γ}-acetyldiaminobutyrate, N_{α}-acetyldiaminobutyrate, histidine, isoleucine, leucine, methionine, phenylalanine, serine, threonine and tyrosine. L-amino acids are preferred. Amino-acid radicals are derived from the corresponding amino acids. The following amino-acid radicals are preferred: Gly, Ala, Ser, Thr, Val, β-Ala, γ-aminobutyrate, Asp, Glu, Asn, Aln, N_{ε}-acetyllysine, N_{δ}-acetylornithine, N_{γ}-acetyldiaminobutyrate, N_{α}-acetyldiaminobutyrate. The amino acids have been abbreviated in accordance with generally usual notation. The di- or tripeptide radicals are acid amides in terms of their chemical nature, and decompose into two or three amino acids upon hydrolysis. The amino acids in the di- or tripeptide radical are joined to one another by amide bonds. With regard to the manufacture of di- and tripeptide radicals, reference is expressly made to EP 0 671 161 A1 of the Marbert company. Examples of di- and tripeptide radicals may also be inferred from the disclosure of EP 0 671 161 A1.

Examples of C₁ to C₄ alkyl groups in the compounds of the formulas of FIG. 17 and FIG. 18 are methyl, ethyl, n-propyl, isopropyl, n-butyl, isobutyl and tert.-butyl. Preferred alkyl groups are methyl and ethyl; methyl is a particularly preferred alkyl group. Preferred C₂ to C₄ hydroxyalkyl groups are the 2-hydroxyethyl, 3-hydroxypropyl or 4-hydroxybutyl groups; 2-hydroxyethyl is a particularly preferred hydroxyalkyl group.

In an exemplary embodiment, the agents contain these care-providing substances in quantities of from about 0.001 to about 2, preferably from about 0.01 to about 0.5 wt %, based in each case on the entire agent.

Mono- and oligosaccharides can also be used as a care-providing substance in the agents. Both monosaccharides and oligosaccharides, for example raw sugar, milk sugar, and raffinose, can be used. The use of monosaccharides is preferred. Among the monosaccharides, those compounds containing 5 or 6 carbon atoms are in turn more preferred.

Suitable pentoses and hexoses are, for example, ribose, arabinose, xylose, lyxose, allose, altrose, glucose, mannose, gulose, idose, galactose, talose, fucose and fructose. Arabinose, glucose, galactose and fructose are carbohydrates that are preferably used; it is more preferred to use glucose, which is suitable both in the D-(+) or L-(−) configuration or as a racemate. Derivatives of these pentoses and hexoses, such as the corresponding -onic and -uronic acids (sugar acids), sugar alcohols, and glycosides, can also be used. Preferred sugar acids are gluconic acid, glucuronic acid, saccharic acid, mannosaccharic acid, and mucic acid. Preferred sugar alcohols are sorbitol, mannitol, and dulcitol. Preferred glycosides are the methylglucosides.

Because the mono- or oligosaccharides that are used are usually obtained from natural raw materials such as starch, in general they exhibit the configurations corresponding to those raw materials (e.g. D-glucose, D-fructose and D-galactose).

In an exemplary embodiment, mono- or oligosaccharides are contained in the agents in a quantity of from about 0.1 to about 8 wt %, and preferably from about 1 to about 5 wt %, based on the entire agent.

The agent can furthermore contain at least one lipid as a care-providing substance. Lipids suitable for use in the agents are phospholipids, for example soy lecithin, egg lecithin, and kephalins, as well as the substances known by the INCI names Linoleamidopropyl PG-Dimonium Chloride Phosphate, Cocamidopropyl PG-Dimonium Chloride Phosphate, and Stearamidopropyl PG-Dimonium Chloride Phosphate. These are marketed, for example, by the Mona company under the commercial designations Phospholipid EFA®, Phospholipid PTC®, and Phospholipid SV®. In an exemplary embodiment, the agents contain the lipids in quantities of from about 0.01 to about 10 wt %, and preferably of from about 0.1 to about 5 wt %, based on the entire agent.

Oily substances are additionally suitable as a care-providing substance in the agents. Among the natural and synthetic cosmetic oily substances may be listed, for example:
Vegetable oils. Examples of such oils are sunflower oil, olive oil, soybean oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils;
Liquid paraffin oils, isoparaffin oils, and synthetic hydrocarbons, as well as di-n-alkyl ethers having a total of between 12 and 36 carbon atoms, in particular 12 to 24 carbon atoms, such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert.-butyl ether, diisopentyl ether, di-3-ethyldecyl ether, tert.-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, are preferred;
Ester oils. “Ester oils” are to be understood as the esters of C₆ to C₃₀ fatty acids with C₂ to C₃₀ fatty alcohols. The monoesters of fatty acids with alcohols having 2 to 24 carbon atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, as well as industrial mixtures thereof that occur, for example, upon high-pressure cleavage of natural fats and oils, upon oxidation of aldehydes from Roelen oxosynthesis, or upon dimerization of unsaturated fatty acids. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol, and brassidyl alcohol, as well as industrial mixtures thereof that occur, for example, upon high-pressure hydrogenation of industrial methyl esters based on fats and oils or aldehydes from Roelen oxosynthesis, and as a monomer fraction upon dimerization of unsaturated fatty alcohols. Particularly preferred are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), Oleyl Oleate (Cetiol®), lauric acid hexyl ester (Cetiol® A), di-n-butyl adipate (Cetiol® B), myristyl myristate (Cetiol® MM), Cetearyl Isononanoate (Cetiol® SN), and oleic acid decyl ester (Cetiol® V);
Dicarboxylic acid esters such as di-n-butyl adipate, di(2-ethylhexyl) adipate, di(2-ethylhexyl) succinate, and diisotridecyl acelaate, as well as diol esters such as ethylene glycol dioleate, ethylene glycol diisotridecanoate, propylene glycol di(2-ethyl hexanoate), propylene glycol diisostearate, propylene glycol dipelargonate, butanediol diisostearate, and neopentyl glycol dicaprylate;
Symmetrical, asymmetrical, or cyclic esters of carbonic acid with fatty alcohols, described for example in German Application DE 197 56 454, glycerol carbonate, or dicaprylyl carbonate (Cetiol® CC);
   Fatty acid triesters of saturated and/or unsaturated linear and/or branched fatty acids with glycerol; and Fatty acid partial glycerides, i.e. monoglycerides, diglycerides, and industrial mixtures thereof. When industrial products are used, small quantities of triglycerides can still be present for manufacturing-related reasons. The partial glycerides preferably conform to formula of FIG. 19, (in which R¹, R² and R³, mutually independently, denote hydrogen or a linear or branched, saturated and/or unsaturated acyl radical having 6 to 22, preferably 12 to 18, carbon atoms, with the stipulation that at least one of these groups denotes an acyl radical and at least one of these groups denotes hydrogen. The sum (m+n+q) denotes 0 or numbers from 1 to 100, preferably 0 or 5 to 25. R¹ preferably denotes an acyl radical and R² and R³ denote hydrogen, and the sum (m+n+q) is 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid, as well as industrial mixtures thereof. Oleic acid monoglycerides are preferably used.

In an exemplary embodiment, the quantity of the natural and synthetic cosmetic oily substances used in the agents is from about 0.1 to about 30 wt % based on the entire agent, preferably about 0.1 to about 20 wt %, and more preferably about 0.1 to about 15 wt %.

The agent can moreover contain an enzyme as a care-providing substance. In a preferred embodiment, the enzyme is selected from a group made up of proteases, lipases, transglutaminase, oxidases and peroxidases.

Pearl extracts are also suitable as a care-providing substance. Mussel pearls are made up substantially of inorganic and organic calcium salts, trace elements, and proteins. Pearls can easily be obtained from cultivated mussels. Mussel cultivation can be accomplished in both fresh water and seawater; this can have an effect on the constituents of the pearls. A pearl extract that derives from mussels cultivated in seawater or salt water is preferred according to the present invention. The pearls are made up largely of aragonite (calcium carbonate), conchiolin, and an albuminoid; the latter constituents are proteins. Also contained in pearls are magnesium and sodium salts, inorganic silicon compounds, and phosphates.

The pearls are powdered for production of the pearl extract. The powdered pearls are then extracted with the usual methods. Water, alcohols, and mixtures thereof can be used as extraction agents for production of the pearl extracts. “Water” is to be understood in this context as both demineralized water and seawater. Among the alcohols, lower alcohols such as ethanol and isopropanol, but in particular polyvalent alcohols such as glycerol, diglycerol, triglycerol, polyglycerol, ethylene glycol, propylene glycol, and butylene glycol, both as a sole extraction agent and also mixed with demineralized water or seawater, are preferred. Pearl extracts based on water/glycerol mixtures have proven to be particularly suitable. Depending on the extraction conditions, the pearl proteins (conchiolin and albuminoid) can be present to a very large extent in the natural state, or already partly or very largely as protein hydrolysates. A pearl extract in which conchiolin and albuminoid are already present in partly hydrolyzed fashion is preferred. The essential amino acids of these proteins are glutamic acid, serine, alanine, glycine, asparagic acid, and phenylalanine. In a further particularly preferred embodiment, it can be advantageous if the pearl extract is additionally enriched with at least one or more of these amino acids. In the most preferred embodiment, the pearl extract is enriched with glutamic acid, serine, and leucine. In addition, depending on the extraction conditions, in particular as a function of the extraction agent selected, a greater or lesser proportion of minerals and trace elements may reappear in the extract. A preferred extract contains organic and/or inorganic calcium salts as well as magnesium and sodium salts, inorganic silicon compounds, and/or phosphates. A preferred pearl extract contains at least 75%, preferably 85%, more preferably 90%, and most preferably 95% of all the constituents of the naturally occurring pearls. Examples of pearl extracts suitable for use in the agents are the commercial products Pearl Protein Extract BG® or Crodarom® Pearl.

In an exemplary embodiment, the pearl extracts described above are contained in the agents in a quantity of from at least about 0.01 to about 20 wt %, preferably from about 0.01 to about 10 wt %, and more preferably from about 0.01 to about 5 wt %, based on the entire agent.

Although each of the aforesaid care-providing substances already yields a satisfactory result of itself, all embodiments in which the agent contains multiple care-providing substances, including from different groups, are also encompassed within the scope of the present invention.

The addition of a UV filter allows both the agent itself and the treated fibers to be protected from damaging influences of UV radiation. At least one UV filter is therefore added by preference to the agent. The suitable UV filters are not subject to any general restrictions in terms of their structure and their physical properties. Instead, all UV filters usable in the cosmetics sector whose absorption maximum lies in the UVA (315-400 nm) UVB (280-315 nm), or UVC (<280 nm) regions are suitable. UV filters having an absorption maximum in the UVB region, in particular in the region from approximately 280 to approximately 300 nm, are particularly preferred.

The UV filters preferred according to the present invention can be selected, for example, from substituted benzophenones, p-aminobenzoic acid esters, diphenylacrylic acid esters, cinnamic acid esters, salicylic acid esters, benzimidazoles, and o-aminobenzoic acid esters.

Examples of UV filters suitable for use in the agents are 4-aminobenzoic acid, N,N,N-trimethyl-4-(2-oxoborn-3-ylidene methyl)aniline methylsulfate, 3,3,5-trimethylcyclohexyl salicylate (Homosalate), 2-hydroxy-4-methoxybenzophenone (Benzophenone-3; Uvinul® M 40, Uvasorb® MET, Neo Heliopan® BB, Eusolex® 4360), 2-phenylbenzimidazole-5-sulfonic acid and its potassium, sodium, and triethanolamine salts (phenylbenzimidazolesulfonic acid; Parsol® HS; Neo Heliopan® Hydro), 3,3′-(1,4-phenylenedimethylene)-bis(7,7-dimethyl-2-oxo-bicyclo-[2.2.1]hept-1-yl-methanesulfonic acid) and its salts, 1-(4-tert.-butylphenyl)-3-(4-methoxyphenyl)propane-1,3-dione (butyl methoxydibenzoylmethane; Parsol®1789, Eusolex® 9020), α-(2-oxoborn-3-ylidene)toluene-4-sulfonic acid and its salts, ethoxylated 4-aminobenzoic acid ethyl ester (PEG-25 PABA; Uvinul® P 25), 4-di-methylaminobenzoic acid 2-ethylhexyl ester (Octyl Dimethyl PABA; Uvasorb® DMO, Escalol® 507, Eusolex® 6007), salicylic acid 2-ethylhexyl ester (Octyl Sa-licylate; Escalol® 587, Neo Heliopan® OS, Uvinul® 018), 4-methoxycinnamic acid isopentyl ester (Isoamyl p-Methoxycinnamate; Neo Heliopan® E 1000), 4-methoxycinnamic acid 2-ethylhexyl ester (Octyl Methoxycinnamate; Parsol® MCX, Escalol® 557, Neo Heliopan® AV), 2-hydroxy-4-methoxybenzophenone-5-sulfonic acid and its sodium salt (Benzophenone-4; Uvinul® MS 40; Uvasorb® S 5), 3-(4′-methylbenzylidene) D,L-camphor (4-methylbenzylidene camphor; Parsol® 5000, Eusolex® 6300), 3-benzylidene camphor (3-Benzylidene Camphor), 4-isopropylbenzylsalicylate, 2,4,6-trianilino-(p-carbo-2′-ethylhexyl-1′-oxi)-1,3,5-triazine, 3-imidazol-4-ylacrylic acid and its ethyl esters, polymers of N-{(2 and 4)-[2-oxoborn-3-ylidenemethyl]benzyl}acrylamide, 2,4-dihydroxybenzophenone (Benzophenone-1; Uvasorb® 20 H, Uvinul® 400), 1,1′-diphenylacrylonitrilic acid 2-ethylhexyl ester (Octocrylene; Eusolex® OCR, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodiumsulfonate, and 2-cyano-3,3-diphenylacrylic acid 2′-ethylhexyl ester. 4-aminobenzoic acid, N,N,N-trimethyl-4-(2-oxoborn-3-ylidene methyl)aniline methylsulfate, 3,3,5-trimethylcyclohexylsalicylate, 2-hydroxy-4-methoxybenzophenone, 2-phenylbenzimidazole-5-sulfonic acid and its potassium, sodium, and triethanolamine salts, 3,3′-(1,4-phenylenedimethylene)-bis(7,7-dimethyl-2-oxo-bicyclo-[2.2.1]hept-1-ylmethanesulfonic acid) and its salts, 1-(4-tert.-butylphenyl)-3-(4-methoxyphenyl)propane-1,3-dione, α-(2-oxoborn-3-ylidene)toluene-4-sulfonic acid and its salts, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and its sodium salt, 3-(4′-methylbenzylidene)-D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-ylacrylic acid and its ethyl esters, and polymers of N-{(2 and 4)-[2-oxoborn-3-ylidenemethyl]benzyl}acrylamide are preferred. Very particularly preferred are 2-hydroxy-4-methoxybenzophenone, 2-phenylbenzimidazole-5-sulfonic acid and its potassium, sodium, and triethanolamine salts, 1-(4-tert.-butylphenyl)-3-(4-methoxyphenyl)propane-1,3-dione, 4-methoxycinnamic acid 2-ethylhexyl ester, and 3-(4′-methylbenzylidene) D,L-camphor.

In a preferred embodiment, the agents comprise those UV filters whose molar extinction coefficient at the absorption maximum is above 15,000, preferably above 20,000.

It has moreover been found that with structurally similar UV filters, in many cases the water-insoluble compound exhibits, in the context herein, the greater effectiveness as compared with water-soluble compounds of this kind that differ from it by having one or more additionally ionic groups. Those UV filters of which no more than 1 wt %, in particular no more than 0.1 wt %, dissolves in water at 20° C. are understood to be water-insoluble. These compounds should furthermore be soluble at a proportion of at least 0.1 wt %, in particular at least 1 wt %, in common cosmetic oil components at room temperature. The use of water-insoluble UV filters are therefore preferred.

According to a further preferred embodiment of the present invention, those UV filters that comprise a cationic group, in particular a quaternary ammonium group, are utilized. These UV filters exhibit the general structure U-Q. The structural part U denotes a group that absorbs UV radiation. This group can in principle be derived from the aforementioned known UV filters usable in the cosmetics sector, in which one group, generally a hydrogen atom, of the UV filter is replaced by a cationic group Q, in particular by a quaternary amino function.

Compounds from which structural part U can be derived include, for example:

substituted benzophenones;

p-aminobenzoic acid esters;

diphenylacrylic acid esters;

cinnamic acid esters;

salicylic acid esters;

benzimidazoles; and

o-aminobenzoic acid esters.

Structural parts U that are derived from cinnamic acid amide or from N,N-dimethylaminobenzoic acid amide are preferred.

Structural parts U can in principle be selected so that the absorption maximum of the UV filters can lie both in the UVA (315-400 nm) region and in the UVB (280-315 nm) region, or in the UVC (<280 nm) region. UV filters having an absorption maximum in the UVB region, in particular in the region from approximately 280 to approximately 300 nm, are particularly preferred. Structural part U is furthermore preferably selected, including as a function of structural part Q, in such a way that the molar extinction coefficient of the UV filter at the absorption maximum is above 15,000, in particular above 20,000.

Structural part Q preferably contains a quaternary ammonium group as a cationic group. This quaternary ammonium group can in principle be connected directly to structural part U, so that structural part U represents one of the four substituents of the positively charged nitrogen atom. Preferably, however, one of the four substituents on the positively charged nitrogen atom is a group, in particular an alkylene group having 2 to 6 carbon atoms, that functions as a connection between structural part U and the positively charged nitrogen atom.

Advantageously, the group Q has the general structure —(CH₂)ₓ—N⁺R¹R²R³X⁻, in which x denotes an integer from 1 to 4, R¹ and R², mutually independently, denote C₁₋₄ alkyl groups, R³ denotes a C₁₋₂₂ alkyl group or a benzyl group, and X⁻ denotes a physiologically acceptable anion. In a preferred embodiment, x denotes the number 3, R¹ and R² each denote a methyl group, and R³ denotes either a methyl group or a saturated or unsaturated, linear or branched hydrocarbon chain having 8 to 22, in particular 10 to 18, carbon atoms. Physiologically acceptable anions are, for example, inorganic anions such as halides, in particular chloride, bromide and fluoride, sulfate ions, and phosphate ions, as well as organic anions such as lactate, citrate, acetate, tartrate, methosulfate, and tosylate.

Two preferred UV filters having cationic groups are the compounds cinnamic acid amidopropyltrimethylammonium chloride (Incroquat® UV-283) and dodecyldimethylaminobenzamidopropyldimethylammonium tosylate (Escalol® HP 610), available as commercial products.

The teaching of the present invention of course also encompasses the use of a combination of several UV filters. In the context of this embodiment, the combination of at least one water-insoluble UV filter with at least one UV filter having a cationic group is preferred.

In an exemplary embodiment, the UV filters are contained in the agents in quantities of from about 0.01 to about 5 wt % based on the entire agent. Quantities from 0.1 to 2.5 wt % are preferred.

In another exemplary embodiment, the agent contains one or more direct-absorbing dyes. This allows the keratinic fibers treated upon application of the agent to be not only temporarily structured, but at the same time also dyed. This can be desirable in particular when what is desired is only temporary coloring (for example, with conspicuous “fashion” colors), which can be removed from the keratinic fibers simply by washing.

Direct-absorbing dyes are usually nitrophenylenediamines, nitroaminophenols, azo dyes, anthraquinones, or indophenols. Preferred direct-absorbing dyes are the compounds known under the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid, and 2-chloro-6-ethylamino-1-hydroxy-4-nitrobenzene.

It is preferred to use cationic direct-absorbing dyes. Particularly preferred in this context are:
a) cationic triphenylmethane dyes such as, for example, Basic Blue 7, Basic Blue 26, Basic Violet 2, and Basic Violet 14;
b) aromatic systems that are substituted with a quaternary nitrogen group, such as, for example, Basic Yellow 57, Basic Red 76, Basic Blue 99, Basic Brown 16, and Basic Brown 17; and
c) direct-absorbing dyes that contain a heterocycle which comprises at least one quaternary nitrogen atom, as recited, for example, in Claims 6 to 11 in EP-A2-998 908, to which reference is explicitly made at this juncture.

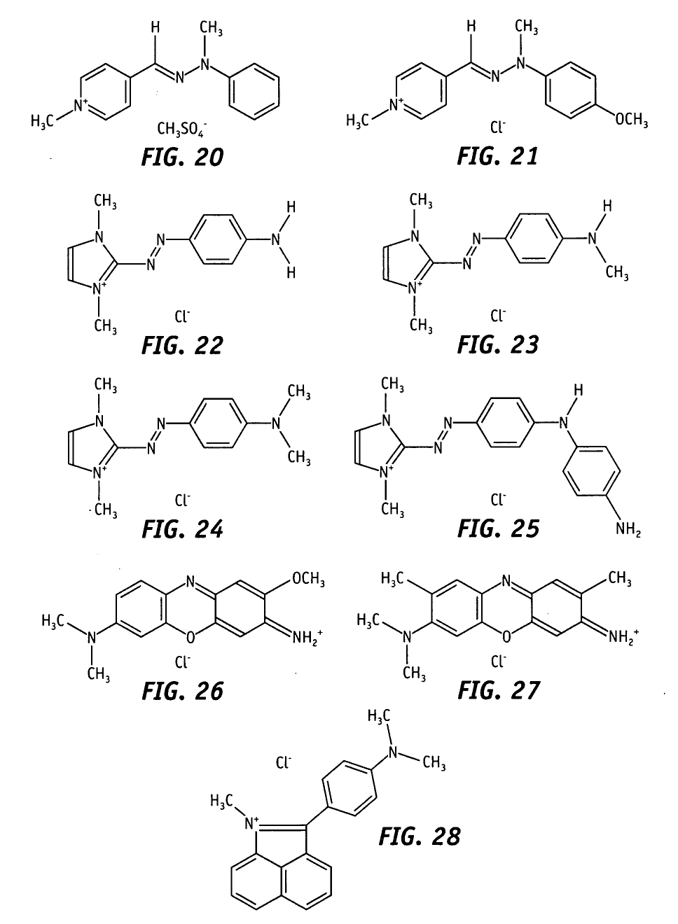

Preferred cationic direct-absorbing dyes of group (c) include, in particular, the compounds of FIGS. 20-28.

The compounds of the formulas of FIGS. 20, 22, and 24, which are also known under the designations Basic Yellow 87, Basic Orange 31, and Basic Red 51, are preferred cationic direct-absorbing dyes of group (c). The cationic direct-absorbing dyes that are marketed under the trademark Arianor® are likewise preferred cationic direct-absorbing dyes.

In an exemplary embodiment the agents contain the direct-absorbing dyes in a quantity of from about 0.001 to about 20 wt %, based on the entire agent.

In addition, the agents can also contain dyes occurring in nature, for example, those contained in henna red, henna neutral, henna black, chamomile blossom, sandalwood, black tea, buckthorn bark, salvia, logwood, madder root, catechu, Spanish cedar, and alkanna root.

It is not necessary for the direct-absorbing dyes to represent homogeneous compounds in each case. The agents can instead, depending on the production methods for the individual dyes, also contain further components in subordinate quantities, provided they do not disadvantageously influence the styling result or do not have to be excluded for other (e.g. toxicological) reasons.

In addition to the aforesaid components, the agents can also contain all active substances, additives, and adjuvants that are known for such preparations. In many cases the agents contain at least one surfactant, both anionic and zwitterionic, ampholytic, nonionic, and cationic surfactants being suitable in principle. In many cases, however, it has proven advantageous to select the surfactants from anionic, zwitterionic, or nonionic surfactants.

Further active substances, adjuvants, and additives include, for example:
thickening agents such as agar-agar, guar gum, xanthan gum, gum arabic, karaya gum, locust bean flour, linseed gums, dextrans, cellulose derivatives, e.g. methyl cellulose, hydroxyalkyl cellulose and carboxymethyl cellulose, starch fractions and derivatives such as amylose, amylopectin, and dextrins, clays such as, for example, bentonite, entirely synthetic hydrocolloids such as, for example, poly(vinyl alcohol) and, if applicable, crosslinked polyacrylates;
structuring agents such as maleic acid and lactic acid;
perfume oils, dimethyl isosorbide, and cyclodextrins;
solvents and solubilizers, such as ethanol, isopropanol, ethylene glycol, propylene glycol, glycerol, and diethylene glycol;
quaternized amines such as methyl-1-alkylamidoethyl-2-alkylimidazolinium methosulfate;
defoamers such as silicones;
dyes for coloring the agent;
anti-dandruff active substances, such as piroctone olamine, zinc olamine, and climbazol;
substances for adjusting the pH, such as, for example, usual acids, in particular edible acids and bases;
cholesterol;
consistency agents such as sugar esters, polyol esters, or polyol alkyl ethers;
fats and waxes, such as spermaceti, beeswax, montan wax, and paraffins;
fatty acid alkanolamides;
complexing agents such as EDTA, NTA, β-alanine diacetic acid, and phosphonic acids;
swelling and penetrating substances, such as glycerol, propylene glycol monoethyl ether, carbonates, hydrogencarbonates, guanidines, ureas, and primary, secondary, and tertiary phosphates;
opacifiers such as latex, styrene/PVP copolymers, and styrene/acrylamide copolymers;
luster agents such as ethylene glycol mono- and distearate, as well as PEG-3 distearate;
preservatives;
stabilizing agents for hydrogen peroxide and other oxidizing agents;
propellants such as propane/butane mixtures, N₂O, dimethyl ether, CO₂, and air; and
antioxidants.

With respect to further optional components as well as the quantities of those components used, reference is explicitly made to the relevant manuals known to those skilled in the art.

Formulation of the agents according to the present invention can be accomplished in all forms that are usual for styling agents, for example in the form of solutions that can be applied onto the hair as a hair lotion or a pump or aerosol spray, in the form of creams, emulsions, waxes, gels, or also surfactant-containing foaming solutions, or other preparations that are suitable for use on the hair.

Hair creams and hair gels generally contain structuring agents and/or thickening polymers which serve to impart the desired consistency to the products. Structuring agents and/or thickening polymers are used typically at a quantity from 0.1 to 10 wt %, based on the entire product. Quantities from 0.5 to 5 wt %, in particular 0.5 to 3 wt %, are preferred.

In various exemplary embodiments, the agents are packaged as a pump spray, aerosol spray, pump hair foam, or aerosol hair foam.

“Hair foams” are understood in this context as compositions that form a foam upon removal from a suitable container. It may be necessary to add to the agents ingredients that promote foam formation or that stabilize a foam once it has formed. Surfactants and/or emulsifiers are especially suitable for this.

If the products according to the present invention involve an aerosol product, the latter may contain a propellant. Propellants suitable for use include, for example, N₂O, dimethyl ether, CO₂, air, and alkanes having 3 to 5 carbon atoms, such as propane, n-butane, isobutane, n-pentane, and isopentane, and mixtures thereof. Dimethyl ether, propane, n-butane, isobutane, and mixtures thereof are preferred.

According to a preferred embodiment, the aforesaid alkanes, mixtures of the aforesaid alkanes, or mixtures of the aforesaid alkanes with dimethyl ether are used as the only propellant. The invention also, however, expressly encompasses the co-utilization of propellants of the fluorochlorohydrocarbon type, but in particular the fluorohydrocarbon type.

For a given spray apparatus, the sizes of the aerosol droplets or foam bubbles, and the respective size distribution, can be adjusted by way of the quantitative ratio of propellant to the other constituents of the preparations. The quantity of propellant used varies as a function of the specific composition of the agent, the packaging used, and the desired type of product (e.g. hair spray or hair foam). When conventional spray apparatuses are used, aerosol foam products contain the propellant preferably in quantities from 1 to 35 wt %, based on the entire product. Quantities from 2 to 30 wt %, in particular from 3 to 15 wt %, are particularly preferred. Aerosol sprays generally contain larger quantities of propellant. In this case the propellant is preferably used in a quantity from 30 to 98 wt % based on the entire product. Quantities from 40 to 95 wt %, in particular from 50 to 95 wt %, are particularly preferred.

The aerosol products can be manufactured in usual fashion. As a rule, all the constituents of the respective agent, with the exception of the propellant, are introduced into a suitable pressure-tight container. The latter is then sealed with a valve. Lastly, the desired quantity of propellant is introduced using conventional techniques.

Particularly preferably, the agents are packaged as an aerosol hair spray. A second subject of the invention is therefore aerosol hair sprays containing the agent and at least one propellant. Preferred agents and propellants of the aerosol hair spray, as well as the respective quantities of the propellant, correspond to what has already been stated above.

A third subject of the invention is the use of the agents according to the present invention for the temporary deformation of keratinic fibers.

The various exemplary embodiments of, the agents, and products, in particular aerosol hair sprays, that contain said agents, are notable in particular for the fact that they impart a very good hairstyle hold to treated hair. They thus exhibit a high degree of hold.

The deformation hold, also referred to as hairstyle hold, as well as the flexibility, elasticity, and plasticity, are determined for purposes herein using the omega loop method. For this, a dry hair strand (European Natural hair of the Kerling company, bonded dense tress, bonded at one end, total length 150 mm, free length 130 mm, width 10 mm, weight 0.9±0.1 g) was immersed for 30 seconds, as far as the upper edge of the adhesive bond, into the polymer solution to be investigated. The excess solution was then wiped off between the thumb and forefinger so that 0.5±0.02 g of solution remained on the hair. The hair strand, saturated with the solution to be investigated, was wound around a Teflon cylinder 36 mm in diameter, and the projecting ends were secured with a clip. The prepared strands were then dried and conditioned in an environmental chamber overnight at 25° C. and 50% relative humidity, or at 25° C. and 75% relative humidity.

The conditioned strand was carefully removed from the Teflon cylinder. The resulting omega loop—a ring-shaped structure of hair stabilized in shape by the polymer film that has formed—was clamped into the grippers mounted on the load cell and lowered to just above the baseplate of an AMETEK LF Plus universal testing instrument of AMETEK Precision Instruments Europe GmbH, Lloyd product group. The entire measurement was performed in an environmental chamber under constant climatic conditions, at 25° C. and 50% relative humidity or at 25° C. and 75% relative humidity.

In order to create standardized initial conditions, the measurement began with application of a preload of 0.07 N at a rate of 30 mm min⁻¹. The omega loop was then compressed 8 mm at a rate of 60 mm min⁻¹, the force necessary therefor being measured. Once the characteristic force F₁ at the maximum deformation of 8 mm had been recorded, the strand was unloaded at 60 mm min⁻¹ until it had risen 10 mm from the baseplate. The next cycle began from there, by once again applying the 0.07 N preload and then compressing the strand 8 mm; the applicable rates were the same as described above. Measurement of one omega loop encompassed a total of 10 cycles.

Four characteristic parameters for describing the mechanical properties of film-forming polymers can be determined using this measurement method. The hold, flexibility, plasticity, and elasticity can be calculated from the measured forces using the following formulas:

Hold=F₁[N]

(F₁ corresponds to maximum measurement force)

$$Flexibility = \frac{{F}_{10}}{{F}_{1}}$$

(indicates the ratio of maximum forces between the tenth and the first cycle)

$$Plasticity = \frac{2 \cdot {H}_{1} - {H}_{10}}{{H}_{1}}$$

(where H₁=9 mm and H₁₀=9 mm+permanent plastic deformation of the strand)

$$Elasticity = \frac{\frac{{F}_{10} ( 2 mm ) - {F}_{10} ( 1.5 mm )}{0.5}}{\frac{{F}_{1} ( 2 mm ) - {F}_{10} ( 1.5 mm )}{0.5}} = \frac{{E}_{10}}{{E}_{1}}$$

(to calculate the elasticity, the forces for a 1.5 mm and 2 mm deformation are acquired respectively from the first and the tenth cycle, and are correlated).

### Examples

The Examples below are intended to explain the subject matter of the present invention without limiting it in any way.

The quantitative indications below are understood, unless otherwise noted, as percentages by weight.

### 1. Aerosol Sprays

The agent E1 according to the present invention, as well as a comparison formulation V1, were produced in accordance with the table below.

| Raw materials | V1 | E1 |
|---|---|---|
| Amphomer ®¹ | 9.60 | 8.70 |
| Allianz LT-120² | — | 1.85 |
| Water, deionized | 12.80 | 11.70 |
| AMP-Ultra PC 1000³ | 1.70 | 1.70 |
| Isopropyl myristate | 0.11 | 0.11 |
| Benzophenone-4⁴ | 0.22 | 0.22 |
| Perfume | 0.11 | 0.11 |
| Ethanol 96%, denatured | to make 100 | to make 100 |

¹N-octyl acrylamide/acrylic acid/tert.-butylaminoethyl methacrylate copolymer (INCI name: Octylacrylamide/Acrylates/Butyl-aminoethyl Methacrylate Copolymer) (National Starch)
²Copolymer of succinic acid C₁ to C₂ alkyl esters, hydroxyalkyl acrylate, and at least one monomer from among acrylic acid, methacrylic acid, and simple esters thereof (approx. 46 to 47.5% solids in water; INCI name: Acrylates/C1-2 Succinates/Hydroxyacrylates Copolymer) (ISP)
³2-Amino-2-methylpropanol (INCI name: Aminomethyl Propanol) (Dow Chemical)
⁴2-Hydroxy-4-methoxybenzophenone-5-sulfonic acid

For the production of aerosol spray products, the agents were respectively introduced into a suitable pressure-tight container that was then sealed with a valve. The agents then had dimethyl ether added to them. The weight ratio of agent to dimethyl ether was 46:54 in each case.

### 2. Verification of Effect

Using the omega loop method (50% and 75% relative humidity), the hold, flexibility, elasticity, and plasticity achievable upon application of a variety of polymers onto human hair were measured. The first investigations were made of polymer solutions P1 and P2, which respectively contained 5 wt % of the film-forming and/or setting polymers Amphomer and Allianz LT-120:

| Raw materials | P1 | P2 |
|---|---|---|
| Amphomer ®¹ | 5.00 | — |
| Allianz LT-120² | — | 10.60 |
| AMP-Ultra PC 1000³ | 0.8 | 0.8 |
| Water, deionized | to make 100 | 40 |
| Ethanol 96%, denatured | — | to make 100 |

The results obtained are reproduced in the table below:

| | Hold (N) | Fleibility | Elasticity | Plasticity |
|---|---|---|---|---|
| P1 (50% RH) | 1.07 | 0.72 | 0.39 | 0.10 |
| P2 (50% RH) | 1.97 | 0.76 | 0.48 | 0.06 |
| P1 (75% RH) | 0.90 | 0.72 | 0.35 | 0.08 |
| P2 (75% RH) | 1.62 | 0.84 | 0.54 | 0.10 |

The results show that as compared with the Amphomer film-forming and/or setting polymer often used in conventional hair sprays, Allianz LT-120 exhibits a definitely higher degree of hold both in an experiment conducted at 50% relative humidity and in an experiment conducted at 75% relative humidity, the flexibility, elasticity, and plasticity being comparable or in fact better.

If however, Amphomer is replaced by Allianz LT-120 in a commercially usual aerosol hair spray, contrary to expectation there is no improvement in the degree of hold. Surprisingly, however, a significant improvement in degree of hold can be achieved when Allianz LT-120 is used in combination with Amphomer.

This is confirmed by investigating the agent E1 according to the present invention and the comparative formulation V1 using the omega loop method (50% relative humidity, 25° C.).

The results obtained are reproduced in the table below:

| | Hold (N) | Fleibility | Elasticity | Plasticity |
|---|---|---|---|---|
| V1 (50% RH) | 2.58 | 0.86 | 0.56 | 0.12 |
| E1 (50% RH) | 2.85 | 0.84 | 0.53 | 0.12 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. An agent for the temporary deformation of keratinic fibers comprising, in a cosmetically acceptable carrier: Oat least one copolymer A formed from:
at least one monomer A 1 selected from the group consisting of acrylic acid, methacrylic acid, acrylic acid alkyl esters, and methacrylic acid alkyl esters;
at least one monomer A2 selected from the group consisting of acrylic acid hydroxyalkyl esters and methacrylic acid hydroxyalkyl esters; and
at least one monomer A3 selected from the group consisting of succinic acid monoalkyl esters and succinic acid dialkyl esters; and
a) at least one film-forming and setting amphoteric polymer B.

2. The agent according to claim 1, wherein the at least one monomer A1 is selected from the group consisting of acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, and methacrylic acid isopropyl ester, and combinations thereof.

3. The agent according to claim 1, wherein the at least one monomer A2 is selected from the group consisting of hydroxymethyl acrylate, hydroxymethyl methacrylate, (2-hydroxyethyl)acrylate, (2-hydroxyethyl)methacrylate, (2-hydroxypropyl)acrylate, (2-hydroxypropyl)methacrylate, (3-hydroxypropyl)acrylate, (3-hydroxypropyl)methacrylate, and combinations thereof.

4. The agent according to claim 1, wherein the at least one monomer A3 is selected from the group consisting of methyl hydrogensuccinate, methyl succinate, ethyl hydrogensuccinate, ethyl succinate, propyl hydrogensuccinate, propyl succinate, isopropyl hydrogensuccinate, and isopropyl succinate, and combinations thereof.

5. The agent according to claim 4, wherein the at least one monomer A3 is selected from methyl succinate and ethyl succinate.

6. The agent according to claim 1, wherein the at least one copolymer A is formed from:
at least one monomer A1 selected from the group consisting of acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, and methacrylic acid isopropyl ester and combinations thereof,
at least one monomer A2 selected from the group consisting of hydroxymethyl acrylate, hydroxymethyl methacrylate, (2-hydroxyethyl)acrylate, (2-hydroxyethyl)methacrylate, (2-hydroxypropyl)acrylate, (2-hydroxypropyl)methacrylate, (3-hydroxypropyl)acrylate, (3-hydroxypropyl)methacrylate, and combinations thereof
at least one monomer A3 selected from the group consisting of methyl succinate and ethyl succinate.

7. The agent according to claim 1, wherein the at least one copolymer A is present in the agent in an amount of from about 0.01 to about 20 wt %, based on the entire hair setting agent.

8. The agent according to claim 7, wherein the at least one copolymer A is present in the agent in an amount of from about 0.05 to about 10 wt %, based on the entire hair setting agent.

9. The agent according to claim 8, wherein the at least one copolymer A is present in the agent in an amount of from about 0.1 to about 5 wt %, based on the entire hair setting agent.

10. The agent according to claim 1, wherein the at least one film-forming and setting amphoteric polymer B is selected from the group consisting of methacryloylbetaine/alkyl methacrylate copolymers, copolymers of monomers having carboxy and/or sulfone groups, in particular acrylic acid, methacrylic acid, itaconic acid, and monomers having amino groups, in particular monoalkylaminoalkyl acrylates, dialkylaminoalkyl acrylates, monoalkylaminoalkyl methacrylates, dialkylaminoalkyl methacrylates, monoalkylaminoalkyl acrylamides, dialkylaminoalkyl acrylamides, monoalkylaminoalkyl methacrylamides, dialkylaminoalkyl methacrylamides, and copolymers of N-octyl acrylamide, methyl methacrylate, hydroxypropyl methacrylate, N-tert.-butylaminoethyl methacrylate, and acrylic acid, and combinations thereof.

11. The agent according to claim 1, wherein the at least one film-forming and setting amphoteric polymer B comprises an N-octyl acrylamide/acrylic acid/tert.-butylaminoethyl methacrylate copolymer.

12. The agent according to claim 1, wherein the at least one film-forming and setting amphoteric polymer B is present in the agent in a quantity of from about 0.01 to about 20 wt %, based on the entire hair setting agent.

13. The agent according to claim 12, wherein the at least one film-forming and setting amphoteric polymer B is present in the agent in a quantity of from about 0.1 to about 15 wt %, based on the entire hair setting agent.

14. The agent according to claim 13, wherein the at least one film-forming and setting amphoteric polymer B is present in the agent in a quantity of from about 1.0 to about 10 wt %, based on the entire hair setting agent.

15. The agent according to claim 1, wherein the at least one copolymer A and the film-forming and setting amphoteric polymer B are present at a weight ratio of from about 1:20 to about 1:1.

16. The agent according to claim 15, wherein the at least one copolymer A and the film-forming and setting amphoteric polymer B are present at a weight ratio from about 1:20 to about 1:2.

17. The agent according to claim 16, wherein the at least one copolymer A and the at least one film-forming and setting amphoteric polymer B are present at a weight ratio of from about 1:20 to about 1:5.

18. The agent according to claim 1, further comprising at least one silicone oil at least one silicone gum, or a combination thereof.

19. The agent according to claim 12, wherein the at least one silicone oil, the at least one silicone gum, or the combination thereof is selected from the group consisting of cyclic and linear polydialkylsiloxanes, alkoxylated and/or aminated derivatives thereof, dihydroxypolydimethylsiloxanes, and polyphenylalkylsiloxanes.

20. A method for using an agent for the temporary deformation of hair, the method comprising the step of applying the agent to hair, wherein the agent comprises, in a cosmetically acceptable carrier:
at least one copolymer A formed from:
at least one monomer A1 selected from the group consisting of acrylic acid, methacrylic acid, acrylic acid alkyl esters, and methacrylic acid alkyl esters;
at least one monomer A2 selected from the group consisting of acrylic acid hydroxyalkyl esters and methacrylic acid hydroxyalkyl esters; and
at least one monomer A3 selected from the group consisting of succinic acid monoalkyl esters and succinic acid dialkyl esters; and
at least one film-forming and setting amphoteric polymer B.

21. An aerosol hair spray comprising:
an agent for the temporary deformation of keratinic fibers comprising, in a cosmetically acceptable carrier:
at least one copolymer A formed from:
at least one monomer A1 selected from the group consisting of acrylic acid, methacrylic acid, acrylic acid alkyl esters, and methacrylic acid alkyl esters;
at least one monomer A2 selected from the group consisting of acrylic acid hydroxyalkyl esters and methacrylic acid hydroxyalkyl esters; and
at least one monomer A3 selected from the group consisting of succinic acid monoalkyl esters and succinic acid dialkyl esters; and
at least one film-forming and setting amphoteric polymer B; and
at least one propellant.
